(19) United States
(12) Patent Application Publication
(10) Pub. No.: US 2009/0076476 A1
(43) Pub. Date: Mar. 19, 2009

(54) SYSTEMS AND METHODS EMPLOYING FORCE SENSING FOR MAPPING INTRA-BODY TISSUE

(75) Inventors: Federico Barbagli, San Francisco, CA (US); Christopher R. Carlson, Menlo Park, CA (US); Neal A. Tanner, Mountain View, CA (US); Christopher M. Sewell, Sunnyvale, CA (US)

Correspondence Address: VISTA IP LAW GROUP LLP, 12930 Saratoga Avenue, Suite D-2, Saratoga, CA, 95070, US

(73) Assignee: Hansen Medical, Inc., Mountain View, CA (US)

(21) Appl. No.: 12/192,974

(22) Filed: Aug. 15, 2008

Related U.S. Application Data
(60) Provisional application No. 60/964,915, filed on Aug. 15, 2007.

(51) Int. Cl. A61B 5/103; A61N 1/00; A61M 31/00

(52) U.S. Cl. 604/500; 600/587; 607/122

(57) ABSTRACT

A medical instrument system includes a controller and a guide instrument coupled to an instrument driver, the instrument driver configured to manipulate a distal end portion of the guide instrument in response to control signals generated by the controller. A force sensor is associated with the guide instrument or with a working instrument carried by the guide instrument, and generates force signals responsive to a force applied to a respective distal end portion of the guide instrument or working instrument. A position determining system generates position data indicative of a position of the respective guide or working instrument distal end portion associated with the force sensor, and a processor operatively coupled to the force sensor and position determining system processes respective force signals and position data to generate and display a geometric rendering of an internal body tissue surface based on sensed forces applied to the respective instrument distal end portion as the guide instrument is maneuvered within an interior region of a body containing the body surface.

### RELATED APPLICATION DATA

The present application claims the benefit under 35 U.S.C. § 119 to U.S. Provisional Patent Application Ser. No. 60/964,915, filed on Aug. 15, 2007. The present application is also related to U.S. patent application Ser. No. 12/150,109, filed on Apr. 23, 2008. The foregoing applications are hereby incorporated by reference into the present application in its entirety.

### FIELD OF INVENTION

The invention relates generally to minimally-invasive instruments and systems, such as manually or robotically steerable catheter instrument systems, and more particularly to systems and methods for sensing, mapping and displaying intra-body tissue compliance.

### BACKGROUND

Standard surgical procedures typically involve using a scalpel to create an opening of sufficient size to enable a surgical team to gain access to an area in the body of a patient for the surgical team to diagnose and treat one or more target sites. When possible, minimally invasive surgical procedures may be used instead of standard surgical procedures to minimize physical trauma to the patient and reduce recovery time for the patient to recuperate from the surgical procedures. Minimally invasive surgical procedures typically require using extension tools (e.g., catheters, etc.) to approach and address the target site through natural pathways (e.g., blood vessels, gastrointestinal tract, etc.) from a remote location either through one or more natural body orifices or percutaneous incisions. As can be appreciated, the surgeon may have limited feedback (e.g., visual, tactile, etc.) to accurately navigate the extension tools, such as one or more catheters, and place the working portions of the extension tools at precise locations to perform the necessary diagnostic and/or interventional procedures. As such, standard surgical procedures might be chosen for the patient even though minimally invasive surgical procedures may be more effective and beneficial for treating the patient.

For example, many conventional minimally-invasive cardiac diagnostic and/or interventional techniques involve accessing the right atrium of the heart percutaneously with a catheter or catheter system (whether manual or robotically controlled) by way of the inferior vena cava. When manually controlling an elongate instrument, such as a catheter, in any one of these applications, the physician operator can push on the proximal end of the catheter and attempt to feel the distal end make contact with pertinent tissue structures, such as the walls of the heart. Some experienced physicians attempt to determine or gauge the approximate force being applied to the distal end of a catheter due to contact with tissue structures or other objects, such as other instruments, prostheses, or the like, by interpreting the loads they tactically sense at the proximal end of the inserted catheter with their fingers and/or hands. Such an estimation of the force, however, is quite challenging and imprecise given the generally compliant nature of many minimally-invasive instruments, associated frictional loads, dynamic positioning of the instrument versus nearby tissue structures, and other factors.

Accordingly, there is a need to develop systems and methods that would facilitate more accurate navigation of extension tools and more precise placement of tools and instruments at target sites for performing diagnostic and/or interventional procedures in minimally invasive operations.

### SUMMARY OF THE DISCLOSED INVENTIONS

Embodiments of the present invention are directed to the use of a robotically-controlled medical instrument system for generating a geometric mapping of an area of internal body tissue (e.g., the wall of a heart chamber), which depicts or is otherwise is correlated to tissue compliance, or a characteristic related to the tissue compliance. In various embodiments, a graphic image or model of the area of body tissue can be generated and/or displayed, with regions of the area differentiated based upon the measured tissue compliance or a characteristic of the tissue that is determined based upon the measured tissue compliance. By way of non-limiting example, the tissue compliance may be used to determine tissue type, such as bone, soft tissue, myocardial wall, etc. In one embodiment, a graphically rendered image of the map depicts a geometric map of the tissue area (e.g., a chamber of the heart), with corresponding respective tissue types displayed in a different color, shade, or other demarcation as determined from their respective compliance.

In one embodiment, a robotically-controlled medical instrument system includes an elongate flexible guide instrument coupled to an instrument driver. The guide instrument defines a working lumen or channel through which an electrophysiology (e.g., mapping and/or ablation) catheter may be positioned through a proximal end opening of the guide instrument in communication with the working lumen. The catheter is inserted through the length of the guide instrument lumen, until a distal end of the catheter extends out of a distal opening of the guide instrument in communication with the lumen. The guide instrument is inserted into a patient's body (the catheter may be inserted into the guide instrument before or after it has been inserted into the body), with a bendable distal end portion of the guide instrument positioned in a selected anatomical workspace to be mapped (or for which a wall portion or other tissue structure is to be mapped). The distal end portion of the guide instrument is maneuvered within the workspace, so that the distal end of the catheter periodically contacting a tissue structure or surface within or bordering the workspace. A force sensor or sensing apparatus associated with the distal end portion of the catheter, e.g., embedded in the distal tip, or coupled to a proximal end of the catheter (i.e., proximal of the guide instrument), senses a force (or “load”) met by the catheter when it comes into contact with the tissue wall or structure. In alternate embodiments, the force sensor may take on numerous different configurations and can be positioned at various locations along the catheter (e.g., built into the tip, or a strain gage provided in a wall of the catheter), such as a load sensor, pressure sensor or other suitable sensor located at or near the distal end of the guide catheter. The force sensor generates force signals responsive to the force applied to the distal end of the guide catheter when it contacts a tissue surface.

The instrument system further includes or is otherwise operatively coupled with a localization (or “position determining”) system for determining the relative position of the distal end of the catheter as it contacts a tissue surface or structure. The position determining system generates position signals which are responsive to the position of the catheter as it is moved to a plurality of locations on an area of body tissue. The position determining system may be any suitable system, including without limitation, localization systems such as those which use magnetic sensors and antenna, open loop or closed loop position systems, shape sensing system such as Bragg fiber optic systems, etc.

The position determining system and force sensor are operatively coupled to a suitable processor (e.g., a system controller or associated computer), a well as associated signal conditioning electronics (collectively, “computer assembly”), with is preferably coupled to a graphic display. The computer assembly is configured to receive and process the position data to generate a geometric map of a tissue surface or other structure based on the localization data provided by the position determining system. The computer assembly is also configured to receive and process the force signals and to calculate a relative compliance of the tissue being contacted by the distal end of the catheter at each of the contact locations. The computer assembly can then generate and display a geometric map correlated with the tissue compliance of the tissue at various regions of the area of tissue of interest.

A method of mapping an area of body tissue using the robotic instrument system is described herein. The guide instrument is introduced into a patient's body. Then, the distal end of the guide catheter is robotically maneuvered into contact with a plurality of locations on an area of body tissue at an interventional procedure site. The robotic instrument system may maneuver the distal end to the plurality of locations in an automated manner (e.g., moving around the heart chamber or other anatomical space and automatically collecting position data and tissue compliance data needed to render a map). For example, a robotic catheter configured with a force sensor may be utilized to palpate and map the interior wall of a uterus or kidney. Alternatively, a physician may drive the catheter by giving commands to the robotic instrument system to go to a particular location, and then to move the distal end of the guide catheter into contact with a plurality of locations on the body tissue. This may be with an organ such as the patient's heart or kidney, or other body lumen such as an artery, or any other body structure. As the distal end of the catheter is moved in to contact with each location on the body tissue, the force on the tip and the deflection of the tissue due to the force is sensed by the system in order to determine the tissue compliance of the tissue at each location. At substantially the same time, the position of each of the locations on the body tissue is also determined. This data is then used to generate a geometric map of the body tissue which is representative of the tissue compliance of the tissue.

Further, the tissue compliance may be used to determine other tissue characteristics such as the type of tissue, condition of the tissue, or other characteristic. For example, one region of the tissue may be very elastic or squishy which may be indicative of soft tissue, while another region may be more firm, indicative of muscle tissue or bone. The method may also be used to identify tissue abnormalities in particular being mapped. For example, a calcified of cancerous tissue can be much harder and less compliant than normal, healthy tissue surrounding it. Then, the generated map can show a graphic image of the area of body tissue with the regions of different tissue characteristics demarcated, such as being shown in different shades, colors, cross-hatching, labels or other suitable graphic indication. The map may then be used in planning and performing a surgical procedure (including diagnosis and treatment procedures), with the same robotic instrument system or other surgical instruments.

Thus, in one embodiment, a medical instrument system (e.g., a robotic instrument system) includes a controller, an instrument driver in communication with the controller, and an elongate instrument coupled to the instrument driver, the instrument driver configured to manipulate a distal end portion of the instrument in response to control signals generated by the controller. A force sensor is associated with the instrument and generates force signals responsive to a force applied to the distal end portion of the instrument. The force sensor may be coupled to the distal end portion of the instrument, or at some other location, and may be a unidirectional force sensor that senses force applied substantially normal to a longitudinal axis of the instrument, or a multi-directional force sensor. A position determining system is also associated with the instrument, and generates position data indicative of a position of the distal end portion of the instrument. The system includes a processor operatively coupled to the force sensor and position determining system, the processor configured to process the respective force signals and position data to generate a geometric rendering of an internal body tissue surface based at least in part upon sensed forces applied to the distal end of the instrument as it is maneuvered within an interior region of a body containing the body surface.

By way of non-limiting examples, the processor may be configured to determine a characteristic of tissue (e.g., tissue stiffness or compliance) at a location on the tissue surface based on a sensed force applied to the distal end portion of the instrument as the instrument is maneuvered against the tissue surface at the respective location. A display is preferably coupled to the processor for displaying the geometric rendering of an internal body tissue surface, for example, wherein regions of the body tissue area in the map having differences in tissue compliance are visually highlighted. In one such embodiment, the graphic rendering of the tissue surface is generated by identifying a plurality of determined positions of the instrument distal end portion within the interior body region at which a substantially same amount of applied force is detected. In another such embodiment, the graphic rendering of the tissue surface includes a first tissue surface boundary determined based upon a first plurality of locations within the interior body region at which a first substantially same amount of force is detected on the distal end portion of the instrument, and a second tissue surface boundary determined based upon a second plurality of locations within the interior body region at which a second substantially same amount of force greater than the first amount is detected on the distal end portion of the instrument. In this embodiment, the processor may be configured to determine a characteristic of an area of tissue in the tissue surface based on a relative spacing between the first and second surface boundaries.

In one embodiment, a working instrument, such as a mapping and/or ablation catheter, is carried by a robotically-driven guide instrument, wherein the force sensor is configured to generate force signals responsive to a force applied to a distal end portion of the working instrument, and the position determining system generates position data indicative of a position of the distal end portion of the working instrument. In this embodiment, the processor generates and displays (or causes to be displayed) a geometric rendering of an internal body tissue surface based at least in part upon sensed forces applied to the distal end of the working instrument while it is extended out of a distal opening of the guide instrument and maneuvered by the guide instrument within an interior region of a body containing the body surface. By way of non-limiting example, the distal tip portion of the working instrument may be extended out of the distal opening of the guide instrument by one or both of retraction of the guide instrument relative to the working instrument and extension of the working instrument relative to the guide instrument. By way of another non-limiting example, the force sensor may be coupled to a proximal portion of the working instrument that extends proximally out of the guide instrument.

In one embodiment, a method of mapping an area of body tissue includes the acts of maneuvering a distal end portion of an elongate instrument within an interior body region; determining a position of the instrument distal end portion within the body region; and sensing a force applied to the instrument distal end portion at the determined position. These acts are repeated for a multiplicity of determined positions of, and sensed forces applied to, the instrument distal end portion within the interior body region, and the respective determined positions and sensed forces for the multiplicity of determined positions are then processed to generate and display a geometric rendering of an internal body tissue surface located within the interior body region, in particular, based on correlating the sensed forces with those corresponding to contacting a tissue surface.

By way of non-limiting example, processing of the respective determined positions and sensed forces to generate and display a geometric rendering of the internal body tissue surface may comprise identifying a plurality of determined positions of the instrument distal end portion within the interior body region at which a substantially same amount of applied force is detected. In one such embodiment, the geometric rendering of the internal body tissue surface includes a first tissue surface boundary determined based upon a first plurality of locations within the interior body region at which a first substantially same amount of force is detected on the instrument distal end portion, and a second tissue surface boundary generated based upon a second plurality of locations within the interior body region at which a second substantially same amount of force greater than the first amount is detected on the instrument distal end portion. In such an embodiment, a characteristic of an area of tissue in the tissue surface may be determined based on a relative spacing between the first and second surface boundaries.

In one such embodiment, a representation of the interior body region is displayed to facilitate initial positioning of the instrument distal end within the region under the control of the operator prior to obtaining of the multiplicity of determined positions.

In one such embodiment, the controller causes the instrument distal end to be maneuvered along a determined set of trajectories based on physical characteristics of the interior body region, e.g., a heart chamber, an anatomical workspace at least partially surrounding an organ exterior surface, an interior of an organ, or an interior of the gastro-intestinal tract, by way of non-limiting examples.

Methods according to such embodiments may further include determining a characteristic (e.g., relative stiffness or compliance, or a surface tension) of an area of tissue on the tissue surface based on a sensed force or forces applied to the instrument distal end portion as it is maneuvered against the tissue surface area. Such methods may also further include identifying a tissue anomaly in the tissue surface based on a sensed force or forces applied to the instrument distal end portion as it is maneuvered against the tissue surface, wherein approximate boundaries of the tissue anomaly on the tissue surface may be displayed for operator review, diagnosis and/or treatment planning.

In accordance with a further aspect of the disclosed inventions, a medical instrument system includes a controller having a user interface for receiving operator input commands; an instrument driver in communication with the controller; and an elongate instrument coupled to the instrument driver, the instrument driver configured to manipulate a distal end portion of the instrument in response to control signals generated by the controller at least partially in response to received operator commands. A force sensor is associated with the instrument, wherein the force sensor generates force signals responsive to a force applied to the distal end portion of the instrument. A processor is operatively coupled to the force sensor and controller, and configured to process the respective force signals to generate applied force data based at least in part upon sensed forces applied to the distal end of the instrument. A display is coupled to the processor for displaying the applied force data. In one such embodiment, the operator commands include an applied force limit on the instrument.

Methods employing this aspect of the disclosed inventions include a method of diagnosing and/or treating internal body tissue by maneuvering a distal end portion of an elongate instrument against an internal body tissue surface until either (i) sensing that a threshold level of force is being applied by the instrument distal end against the tissue surface, or (ii) the instrument distal end is extended to or beyond a determined movement limitation. Such methods may be carried out using a robotic instrument coupled to an instrument driver configured to manipulate the instrument distal end portion in response to control signals generated by a controller, wherein the control signals being generated at least in part in response to operator commands received through a user interface coupled to the controller.

In one such embodiment, the user interface includes a haptic input device, wherein the controller transmits signals to the input device to cause the input device to impart a detectable resistance to movement of the input device corresponding to an actual amount of force being applied against the instrument distal end portion by the tissue surface. Upon sensing that the threshold level of force is being applied by the instrument distal end against the tissue surface, the controller transmits a workspace limitation signal to the input device causing the input device to prevent movement of the input device in a manner that would cause a corresponding movement of the instrument distal end against the tissue surface and further increase the amount of applied force.

If for some reason the instrument distal end is extended to or beyond the determined movement limitation prior to reaching the threshold force level, the controller at least partially disables the user interface to prevent additional extension of the instrument distal end portion. A graphical representation of the instrument distal end and tissue surface may be displayed in conjunction with the procedure, wherein movement of the instrument distal end relative to the body surface is shown substantially in real time, including a representation of an actual sensed force applied by the instrument distal end against the body surface. By way of example, the graphical representation of the instrument distal end may change in color based on a corresponding change or changes in the actual sensed force applied by the instrument distal end against the body surface.

In accordance with yet another embodiment, a medical instrument system includes an elongate instrument, and a controller configured to selectively actuate one or more motors operatively coupled to the instrument to thereby selectively move the instrument. A force sensor associated with the instrument generates force signals responsive to a force applied to the distal end portion of the instrument, and a processor operatively coupled to the force sensor and controller processes the respective force signals to generate applied force data based at least in part upon sensed forces applied to the distal end of the instrument. The system further includes a haptic input device in communication with the controller and configured for generating instrument motion commands in response to a directional movement of the input device, wherein the controller transmits signals to the input device to cause the input device to impart a detectable resistance to movement of the input device corresponding to an actual amount of force being applied against the instrument distal end portion.

In accordance with still another embodiment, a method of diagnosing and/or treating internal body tissue includes maneuvering a distal end portion of an elongate instrument against an internal body tissue surface, sensing an axial force vector applied by the body surface to the instrument distal end portion, determining an angle of incidence at which the instrument distal end portion is contacting the body surface, and projecting, based on the sensed axial force vector and determined contact angle of incidence, a component of the axial force in a direction normal to the tissue surface at the contact location. Such method may further include projecting a component of the axial force vector in a direction tangential to the tissue surface at the contact location.

Other and further aspects and embodiments are disclosed in the following detailed description, which is to be read in conjunction with the figures.

### BRIEF DESCRIPTION OF THE DRAWINGS

The drawings illustrate the design and utility of illustrated embodiments of the invention, in which similar elements are referred to by common reference numerals, and in which:

FIG. 1 illustrates one embodiment of a robotic instrument system.

FIG. 2 illustrated one embodiment of a catheter assembly used in the robotic instrument system of FIG. 1.

FIG. 3 illustrates a schematic representation of a robotic catheter system including a force sensing system.

FIG. 4 illustrates one embodiment of a guide catheter having a force sensor system.

FIG. 5 illustrates another embodiment of a guide catheter having a force sensor system.

FIG. 6 illustrates a system having an instrument driver and an ablation energy control unit.

FIG. 7 is a simplified schematic diagram of a three-dimensional mapping system shown coupled to a body for mapping a portion of a heart.

FIGS. 8-9 illustrate a distal end portion of a catheter carrying a plurality of localization electrodes and positioned in a respective heart chamber.

FIG. 10 illustrates a distal end portion of a medical instrument assembly located in a heart chamber.

FIG. 11 is a process flowchart for generating a three-dimensional map using force sensing data produced using a robotic instrument system, as well as for optionally verifying and/or calibrating a three-dimensional map or portions of a three-dimensional map produced by a conventional 3-D mapping system.

FIG. 12 is a geometric rendering of a body cavity tissue surface constructed in accordance with one embodiment.

FIG. 13 illustrates the distal end portion of a medical instrument assembly, including coaxial sheath and guide catheters, and a surgical instrument such as a needle or knife carried in the guide catheter, the assembly positioned within an interior region of a body cavity.

FIG. 14 illustrates the views of an anatomy as may be captured by a fluoroscope.

FIGS. 15A and 15B are respective side perspective views of the instrument assembly of FIG. 13 in a body cavity and registered in the anterior posterior view (FIG. 15A) and right anterior view (FIG. 15B), respectively.

FIG. 16 illustrates a distal end portion of an instrument assembly coupled with one or more optical fiber sensors configured with Bragg gratings.

FIGS. 17 and 18 illustrate a plurality of localization (voltage potential) electrodes coupled to an instrument and positioned in a respective heart chamber.

FIG. 19 illustrates a portion of the needle of the instrument depicted in FIG. 17, in which certain sections of the needle are configured to behave or function as electrodes.

FIG. 20 illustrates the concept of deriving a force component normal to a tissue surface when the force sensing instrument is pressed into the surface at a non-orthogonal angle.

FIG. 21 depicts the distal end portion of a robotic guide instrument maneuvering a working instrument within an anatomical workspace during a surface tissue mapping procedure.

FIG. 22 depicts the distal end portion of the respective guide and working instruments during a surface mapping procedure within an interior region of a kidney.

FIG. 23 illustrates respective tissue compliance curves for two distinct tissue surfaces.

FIG. 24 depicts a multi-force level map of a tissue surface circumscribing a body cavity region.

FIG. 25 depicts a process for limiting and stabilizing an amount of applied force.

FIG. 26 depicts a displayed tissue surface map, in which an area of tissue having been identified as having relatively higher or lower compliance/stiffness than the surround tissue surface is visually highlighted.

FIG. 27 illustrates another embodiment of a robotic instrument having both a force sensor and an image capture device in its distal tip.

FIG. 28 illustrates still another embodiment of a robotic instrument system, including a pair of robotic arms extending from an access sheath, one robotic arm having a force sensor and the other robotic arm having an image capture device.

### DETAILED DESCRIPTION OF THE ILLUSTRATED EMBODIMENTS

Robotic interventional systems and devices such as the Sensei™ Robotic Catheter System and the Artisan™ Control Catheter manufactured and distributed by Hansen Medical, Inc., Mountain View, Calif., are well suited for use in performing minimally invasive medical procedures. Exemplary embodiments of robotic instrument systems that may be modified for constructing and using embodiments of the present invention are disclosed and described in detail U.S. patent application Ser. Nos. 11/073,363, filed Mar. 4, 2005, Ser. No. 11/179,007, filed Jul. 6, 2005, U.S. patent application Ser. No. 11/418,398, filed May 3, 2006, U.S. patent application Ser. No. 11/481,433, filed Jul. 3, 2006, and U.S. patent application Ser. No. 11/640,099, filed Dec. 14, 2006, which are all incorporated herein by reference in their entirety. Additionally, U.S. Patent Publication 2007/0233044 (the “'044 publication”), which is incorporated herein by reference in its entirety, discloses embodiments of such robotically-navigated interventional systems, including the capability to sense force between a surface of an internal body cavity or lumen (referred to collectively as a “body space”) and a distal end of a working instrument (e.g., an ablation catheter) carried in a working lumen of a robotically controlled guide instrument. The system not only detects contact between the instrument and the surface, but also measures the magnitude of the force, also called the load. In particular, the system can also be used to detect contact with tissue structures due to the change in the sensed force. Force sensing and force feedback capabilities may be provided by an Artisan™ Control Catheter and IntelliSense™ Fine Force Technology™ provided on the Sensei™ Robotic Catheter System manufactured and distributed by Hansen Medical, Inc.

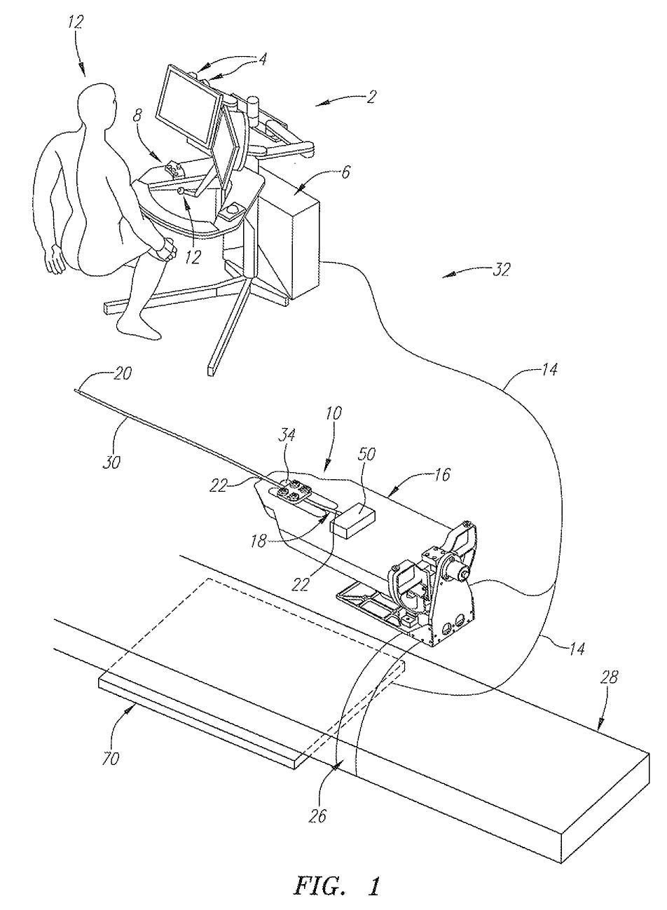

One illustrative embodiment of a robotic instrument system (32) according to the present invention is shown in FIG. 1. The robotic instrument system (32) includes an operator control station (2) located remotely from an operating table (22), and a robotic catheter assembly (10). The control station (2) comprises a user interface (8) that is operatively connected to the robotic catheter assembly (10). A physician or other operator (12) interacts with the user interface (8) to operate the robotic catheter assembly (10). The user interface (8) is connected to the robotic catheter assembly (10) via a cable (14) or the like, thereby providing one or more communication links capable of transferring signals between the operator control station (2) and the robotic catheter assembly (10). Alternatively, the user interface (8) may be located in a geographically remote location and communication is accomplished, at least in part, over a wide area network such as the Internet. Of course the user interface (8) may also be connected to the robotic catheter assembly (10) via a local area network or even wireless network that is not located at a geographically remote location.

The control station (2) also comprises a display (4) that is used to display various aspects of the robotic instrument system (2). For example, an image of the working instrument and guide instrument (described in further detail below) may be displayed in real time on the display (4) to provide the physician (12) with the current orientation of the various devices as they are positioned, for example, within a body lumen or region of interest. The control station (2) further comprises a computer assembly (6), which may comprise a personal computer or other type of computer work station for performing the data processing operations disclosed herein. The robotic catheter assembly (10) is coupled to the operating table (22) by an instrument driver mounting brace (26). The robotic catheter assembly (10) comprises a robotic instrument driver (16), a working catheter (18), and a guide catheter (30) (also referred to herein as an instrument guide catheter, guide catheter, robotic guide instrument, robotic guide catheter, or the like). The instrument driver mounting brace (26) of the depicted embodiment is a relatively simple, arcuate-shaped structural member configured to position the instrument driver (16) above a patient (not shown) lying on the table (22).

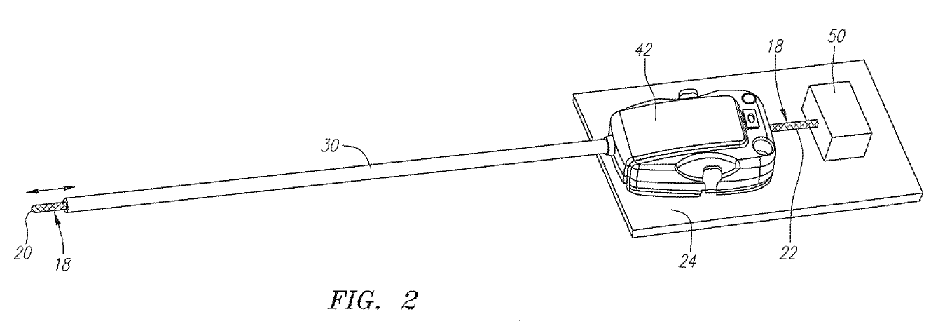

Referring to FIG. 2, the catheter (18) is typically an elongate, flexible device configured to be inserted into a patient's body. The catheter (18) has a distal end (20) and a proximal end (22). As non-limiting examples, the catheter (18) may comprise an intravascular mapping and/or ablation catheter, an endoscopic surgical instrument or other medical instrument. The catheter (18) is configured to be operable via the instrument driver (16) such that the instrument driver (16) can operate to steer the catheter (18) and also to operate tools and devices (also called end effectors) which may be provided on the instrument assembly (18) (e.g. an imaging device or cutting tool disposed on the distal end of the catheter (18). The working catheter (18) may be movably positioned within the working lumen of the guide catheter (30) to enable relative insertion of the two instruments, relative rotation, or “roll” of the two instruments and relative steering or bending of the two instruments relative to each other, particularly when the distal end (20) of the working catheter (18) is inserted beyond the distal tip of the guide catheter (30). The system (32) also comprises a mechanical ditherer (50) or other dithering mechanism or device as described herein for sensing and measuring forces applied to the distal end (20) of the catheter (18).

The guide catheter (30) is mounted via a base (24) carrying the ditherer (50). The ditherer (50) is coupled to the working catheter (18) that is dithered back-and-forth relative to the guide catheter (30). The guide catheter (30) is coupled to housing (42) that mechanically and electrically couples the guide catheter (30) to a robotically-controlled manipulator. For example, the guide catheter (30) may be coupled to a robotically controlled instrument driver such as, for instance, the above-mention Sensei™ Robotic Catheter System manufactured and distributed by Hansen Medical.

Referring back to FIG. 1, the system (32) also comprises a force sensor system (34) for sensing the force on the distal end (20) of the working catheter (18). The force sensor system (34) may be disposed at various locations, including a force sensor placed on the distal end (20) of the working catheter (18), a force sensor placed on the proximal end (22) of the working catheter (18), a ditherer force sensor system (as described in more detail below) located at the proximal end of the working catheter (18), or other suitable location. The force sensor system (34) is in operable communication with the operator control station (2) via the communication link (14). In alternative embodiments, one or more force sensors (not shown) may be coupled to and/or embedded within various locations on the catheter (18), for example in the distal end (20). Such force sensor(s) may be unidirectional force sensors, for example, a unidirectional force sensor positioned in the center of the catheter end (30) for sensing force applied substantially normal to a longitudinal axis of the catheter (18). Alternatively and/or additionally, such force sensor(s) may be arranged to sense multi-directional forces (e.g., in orthogonal x, y, z planes).

As is described in greater detail herein, the system (32) further comprises a position determining system (70) for determining the position of the distal end (20) of the working catheter (18). The position determining system (70) may be any suitable localization system, many of which are commercially available, including without limitation localization systems that use magnetic or voltage potential sensors, such as the Carto™ XP available from Biosense Webster, Inc. (a subsidiary of Johnson & Johnson), the EnSite NavX™ available from St. Jude Medical, and the microBird™ available from Ascension Technology, each of which are which are capable of sensing the relative locations of each of a plurality of sensors (72) located on the catheter (18). The position sensing system (70) may also be a shape sensing system that employs fiber optic Bragg grating shape sensing, such as systems disclosed in U.S. patent application Ser. Nos. 11/690,116, filed Mar. 22, 2007, Ser. No. 12/106,254, filed Apr. 18, 2008, and Ser. No. 12/192,033, filed Aug. 14, 2008, and in U.S. Provisional Patent Application No. 61/003,008, filed Nov. 13, 2007, which are all incorporated herein by reference in their entirety. The position determining system (70) is in operable communication with a computer assembly (6) of the operator control station (2) through the communication link (14). The computer assembly (6) may comprise conditioning electronics for conditioning the force signals from the force sensor system (34) and the position signals from the position determining system (70).

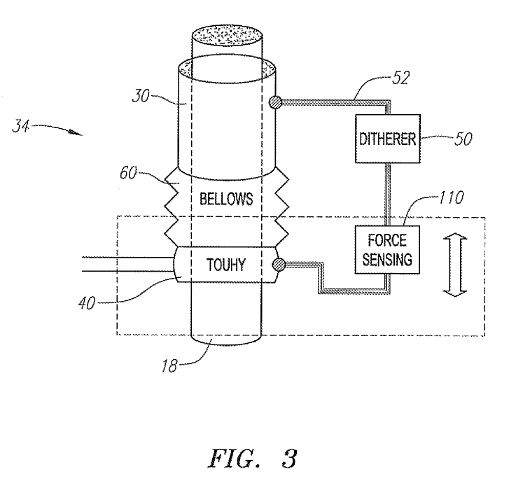

Turning now to FIG. 3, one embodiment of a force sensor system (34) intended to be located at the proximal end (22) of the catheter (18) will be described. FIG. 3 is a schematic illustration of a system and method for measuring a force on the distal end (20) of the catheter (18) using a dithering technique. In this embodiment, the working catheter (18) dithers with respect to substantially stationary guide catheter (30). In order to dither the working catheter (18) back and forth (longitudinally), the mechanical ditherer (50) will drive the working catheter (18) through a force sensor (110), which will measure the direct force needed to insert and withdraw the working catheter (18) in and out of the guide catheter (30). The ditherer (50) is mechanically grounded (via a mechanical linkage 52) to a proximal region of the guide catheter (30) and is thus stationary relative to the guide catheter (30), but the force sensor (110) and working catheter (18) move together relative to the guide catheter (30). The force signals from the force sensor (110) are transmitted to the computer assembly (6) for data processing. This type of force sensor system (34) is described in detail in the above-incorporated '044 publication, along with various other embodiments of “dithering” force sensors.

In the embodiment illustrated in FIG. 3, the ditherer (50) and force sensor (110) are mechanically linked to a seal (40), such as a Touhy seal. The Touhy seal (40) acts as a fluidic seal which can add significant and erratic drag to the reciprocating in-and-out motion of the working catheter (18), which would adversely affect the accuracy of readings from the force sensor (110). This embodiment eliminates this effect by mechanically securing or locking the Touhy seal (40) to the working catheter (18) so the two are dithered together. In addition, FIG. 3 illustrates the flexible bellows (60) that is connected to the proximal end of the guide catheter (30) at one end and secured to the Touhy seal (40) at the other end. The bellows (60) expands and contracts like an accordion with the dithering motion. The bellows (60) advantageously applies a very low drag force on the working catheter (18) during the dithering motion as opposed to the high drag force that would be applied if the working catheter (18) was dithered through the Touhy seal (40).

By “dithering” the working catheter (18) with respect to the guide catheter (30), the repeated cyclic motion may be utilized to overcome frictional challenges normally complicating the measurement, from a proximal location, of loads at the distal end (20) of the working catheter (18) when in contact with a surface. In one embodiment, the dithering motion may be applied on a proximal region of the working catheter (18) as is illustrated in FIG. 1. In other words, for example, if an operator were to position a working catheter (18) down a lumen of a guide catheter (30) so that the distal end (20) of the working catheter (18) is sticking out slightly beyond the distal end of the guide catheter (30) (as shown in FIG. 2), and have both the guide catheter (30) and working catheter (18) advanced through the blood vessel(s) from a femoral location to the chambers of the heart, it may be difficult to sense contact(s) and force(s) applied to the distal end (20) of the working catheter (18) due to the complications of the physical relationship with the associated guide catheter (30). In particular, in a steady state wherein there is little or no relative axial or rotational motion between the working catheter (18) and guide catheter (30), the static coefficient of friction is applicable, and there are relatively large frictional forces keeping the working catheter (18) in place relative to the guide catheter (30) (no relative movement between the two).

To release this relatively tight coupling and facilitate proximal measurement of forces applied to the distal end (22) of the working catheter (18), dithering motion may be used to effectively break loose this frictional coupling. In the embodiment illustrated in FIG. 3, the dithering motion may be applied on a proximal region of the working catheter (18). In alternative embodiments (not shown), it may be possible to dither the guide catheter (30) with respect to a stationary or substantially stationary working catheter (18). In yet another embodiment, both the working catheter (18) and guide catheter (30) may be dithered with respect to one another. Notably, while the embodiment illustrated in FIG. 3 shows the operation of the ditherer 50 with respect to a catheter assembly that includes both a working catheter and a guide catheter, it should be appreciated that the functionality of the working and guide catheters can be incorporated into a single catheter to which the ditherer 50 is operatively coupled.

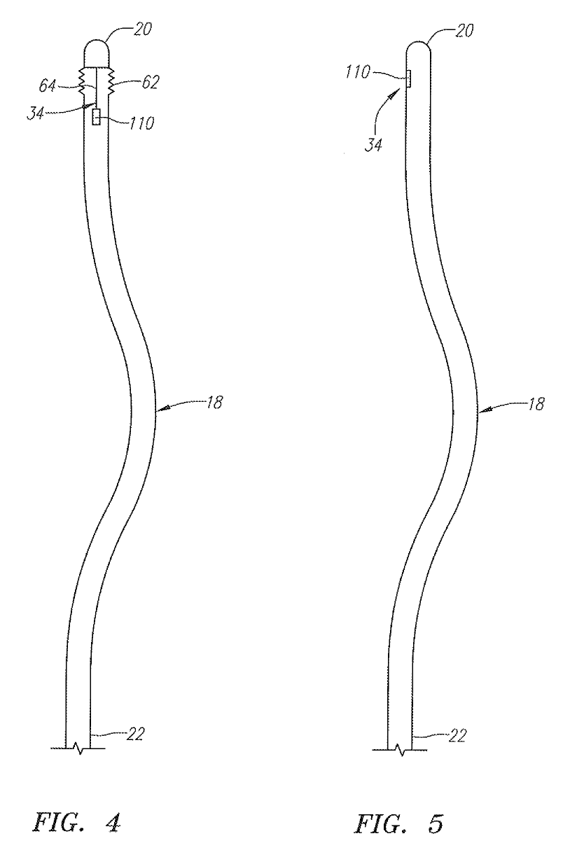

The issues presented by the frictional forces and other complexities associated with a force sensor located at the proximal end (22) of the working catheter (18) may be eliminated by locating the force sensor at or near the distal end (20) of the working catheter (18). FIG. 4 and FIG. 5 illustrate two exemplary embodiments. Referring first to the embodiment of FIG. 4, a force sensor system (34) is located at or near the distal end (20) of the working catheter (18). The force sensor system (34) comprises a flexible bellows (62) that expand and contract in response to a force placed on the distal end (22). A transfer rod (64) is coupled at one end to the distal end (22) and at the other end to a force sensor (110). The force sensor (110) may be any suitable force sensor such as a load sensor, pressure sensor, piezoelectric sensor, strain gauge or the like. When the distal end (22) contacts body tissue, the bellows is compressed causing the transfer rod (64) to push on the force sensor (110). The force sensor (110) transmits a force signal responsive to the amount of force being applied to the distal end (22) to the computer assembly (6).

The working catheter (18) of FIG. 5 is similar to that of FIG. 4, except that the force sensor system (34) is applied directly onto the shaft or even the distal end (20) of the working catheter (18). The force sensor system (34) comprises a force sensor (110), which transmits a force signal to the computer assembly (6). In this embodiment, the strain of the working catheter (18) itself is used to measure the force being applied to the distal end (18). Although each of the different types of force sensors (110) described herein may be utilized, a strain gauge may be the most suitable for this embodiment.

As briefly discussed above, the computer assembly (6) is configured to receive and process the force signals from the force sensor system (34) and the position signals from the position determining system (70). It should be understood that the computer assembly (6) may comprise one or more computers, signal conditioning electronics, and other displays and peripherals. The computer assembly (6) is also configured to process the force signals and position signals to generate a geometric map of an area of body tissue correlated to the tissue compliance of the tissue or other characteristic of the body tissue related to its tissue compliance.

As an example, as the working catheter (18) is robotically maneuvered within a patient's body at an area of interest, the distal end (20) is moved into contact with the plurality of locations on the area of body tissue. The computer assembly (6) receives the position signals and force signals and determines the force on the tip, the deflection of the tissue and the position of the location on the area of body tissue at each of the plurality of locations. The computer assembly (6) is further configured to generate a geometric map of the area of body tissue using the position determined for each location, and to also correlate the tissue compliance at each location and superimpose the tissue compliance on the geometric map. Regions of different compliance may be superimposed on the mapping in different colors, shades or other suitable representation. The computer assembly (6) may also be configured to relate the measured compliance of the different regions of the area of tissue to other tissue characteristics, such as tissue type, tissue condition (necrosed, healthy, diseased, etc.) or other characteristic of interest.

This approach is similar to that described in U.S. Pat. No. 5,391,199 to Ben-Haim et al. (the “'199 patent”), which is incorporated herein by reference in its entirety. The '199 patent discloses methods of detecting contact of the instrument tip with a body surface in combination with localization techniques to generate a graphic, geometric representation or “map” of a body structure, such as the surface surrounding a body lumen or cavity (e.g., a heart chamber). The '199 patent describes a geometric mapping of the walls of a body lumen or cavity using a manual catheter by sensing contact with a plurality of locations on the surface(s) of the lumen or cavity and using localization sensors to determine position coordinates of the instrument tip at each of the plurality of locations. This position data is then used to construct a geometric map of the body lumen or cavity.

Returning to the embodiments of FIGS. 1-5, the robotic instrument system (32) may maneuver the distal end to the plurality of locations in an automated manner in response to a programmed path or target (e.g., moving around the heart chamber or other anatomical space and automatically collecting position data and tissue compliance data needed to render a map). Alternatively, a physician may drive the working catheter (18) by giving commands to the robotic instrument system (32) to move the working catheter (18) to a particular location, and then to move the distal end (20) of the working catheter (18) into contact with a plurality of locations. As an example, the human heart is composed of three primary types of tissue: the myocardium which is the muscular tissue of the heart; the endocardium which is the inner lining of the heart; and the epicardium which is a connective tissue layer around the heart. These tissues have inherently differing elastic properties, e.g. the myocardium tissue is firmer than the endocardium tissue. Accordingly, to map a patient's heart, the working catheter (18) can be advanced into the heart of a patient, the distal end (20) is then contacted with a plurality of locations within the heart, and a map can be generated showing an image of the different structures of the heart.

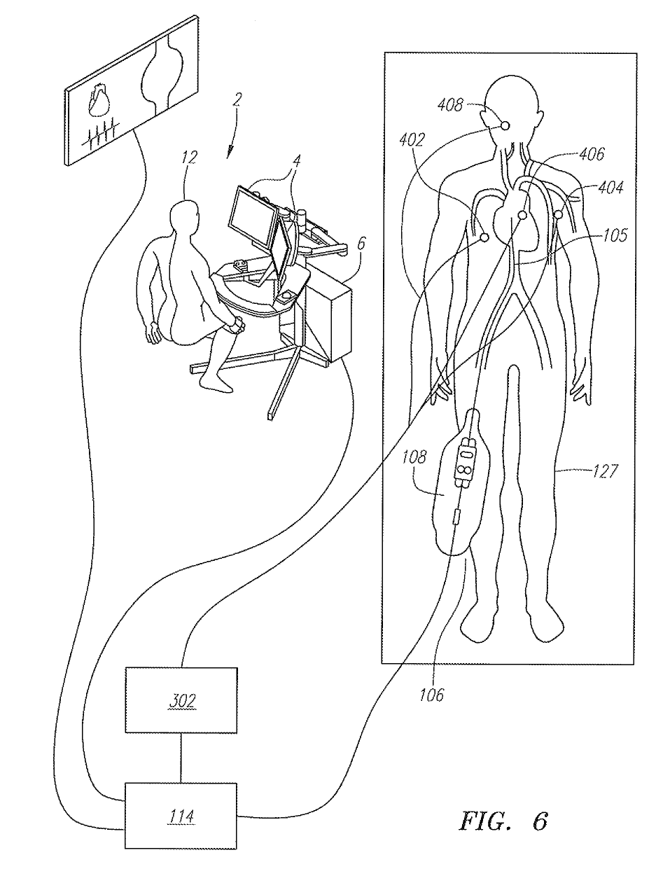

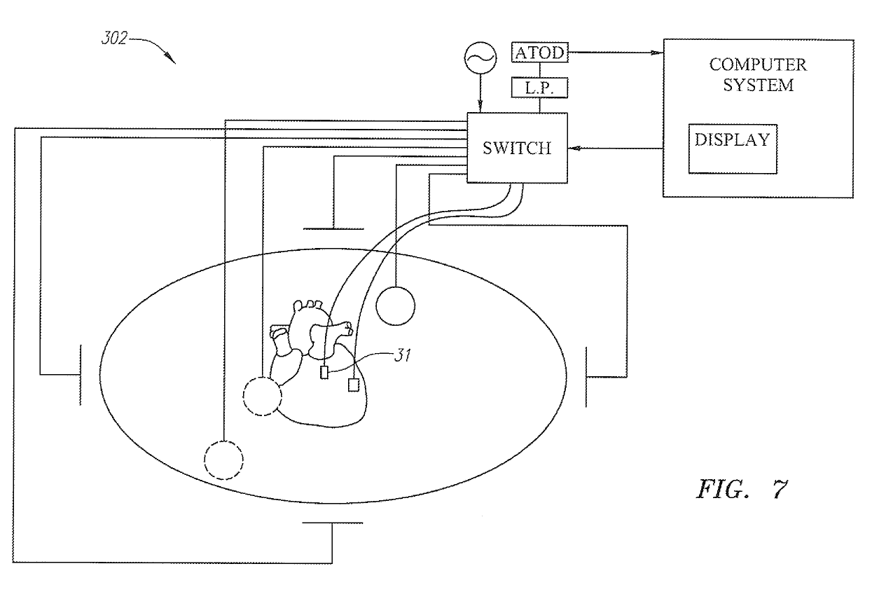

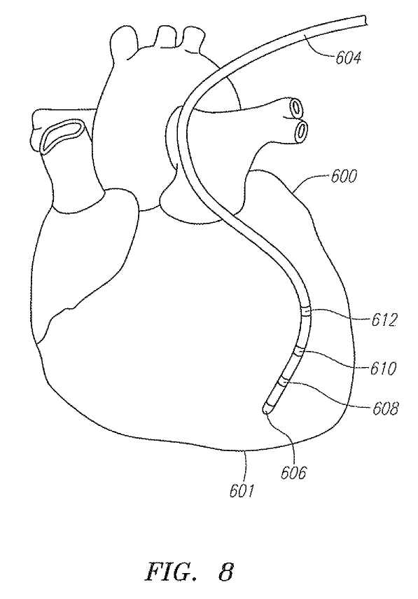

FIG. 6 illustrates a schematic embodiment of a robotic surgical system (114) being used to perform minimally invasive surgery using one or more instrument assemblies (108). The illustrated instrument assembly (108) includes one or more independently movably controlled (e.g., maneuvered, steered—pitch, yaw, rotate, etc., advanced, etc.) catheters (105), e.g., sheath catheter, guide catheter, etc. The robotic surgical system (114) includes a three-dimensional (3-D) mapping system (302), which may be may be similar to the EnSite NavX™ Navigation & Visualization Technology available from St. Jude Medical, Inc. In particular, the 3-D mapping system (302) is configured to produce a three-dimensional map of interior spaces of body cavities or volumes (e.g., a body cavity might be an organ such as a heart, stomach, uterus, bladder, etc.) from modulated electrical fields. As illustrated in FIG. 6, the modulated electrical fields may be generated by a combination of electrodes (402, 404, 406, 408, etc.) positioned on the body of a patient 127.

The 3-D mapping system (302) is capable of producing diagnostic data using time domain and frequency domain representations of electrophysiology data. Exemplary maps include time domain difference between action potentials at a roving electrode (e.g., an electrode coupled to a catheter) and a reference electrode; the peak-to-peak voltage of action potentials at the roving electrode; the peak negative voltage of action potentials at the roving electrode; complex fractionated electrogram information; a dominant frequency of an electrogram signal; a maximum peak amplitude at the dominant frequency; a ratio of energy in one band of the frequency-domain to the energy in a second band of the frequency-domain; a low-frequency or high frequency passband of interest; a frequency with the maximum energy in a passband; a number of peaks within a passband; an energy, power, and/or area in each peak; a ratio of energy and/or area in each peak to that in another passband; and a width of each peak in a spectrum. Colors, shades of colors, and/or gray scales are assigned to values of the parameters and colors, shades of colors, and/or gray scales corresponding to the parameters for the electrograms sampled by the electrodes are provided and updated on the three-dimensional map or model. One example of such a 3-D mapping system (302) is described in U.S. Patent Publication 2007/0073179, filed Sep. 15, 2005, the contents of which are fully incorporated herein by reference.

FIG. 7 illustrates a schematic diagram of one embodiment of a three-dimensional mapping system (302). The 3-D mapping system (302) may use up to sixty-four electrodes in and/or around a body cavity or organ, such as a heart and the vasculature of a patient, measure electrical activity at up to sixty-two of those sixty-four electrodes, and produce a three-dimensional map of the time domain and/or frequency domain information from the measured electrical activity (e.g., electrograms) for a single beat of the heart. The number of electrodes capable of being simultaneously monitored is limited by the number of electrode lead inputs into the system (302) and the processing speed of the system (302). The electrodes may be stationary or may be moving (e.g., some electrodes may be attached to a roving catheter).

In one embodiment, illustrated in FIG. 8, a catheter (604) carrying a plurality of electrodes (606, 608, 610, 612) on a distal end portion thereof may be extended into the left ventricle (601) of the heart (600). The electrodes (606, 608, 610, 612) may be in direct contact with the wall surface of the heart (600) or they may be generally near or adjacent to the wall surface of the heart (600) to measure and collect information on the electrical activity of the heart.

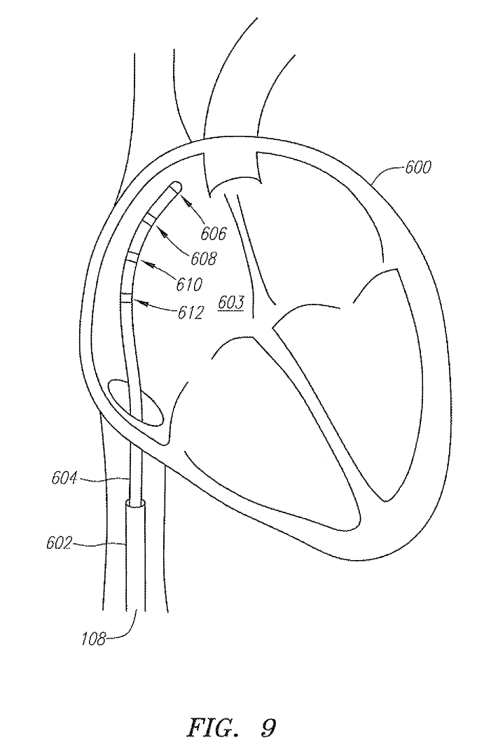

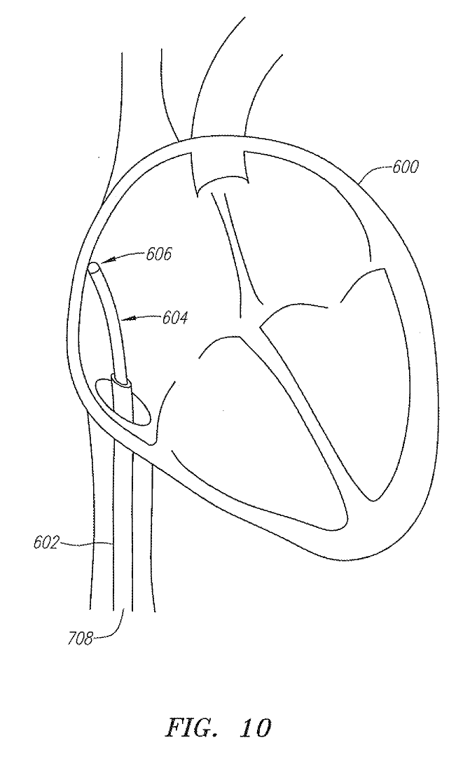

FIG. 9 illustrates a catheter (604) extended into the right atrium (603) of the heart (600), where data points may be taken to produce a three-dimensional map. In addition to the above-referenced '199 patent, an exemplary positioning system for determining the position or location of a catheter in the heart is described in U.S. Pat. No. 5,697,377, which is incorporated by reference herein in its entirety. In another embodiment, an array of electrodes may be used. For example, an array of electrodes may be coupled to a catheter to collect real-time cardiac electrical data for producing a three-dimensional map or model (e.g., an isopotential map or model). In such an embodiment, the positioning system may produce or determine electrograms for up to about three thousand locations along the wall or wall surface of the heart. Such a system employing an array of electrodes is described in U.S. Pat. No. 5,662,108, which is incorporated by reference herein in its entirety. A similar implementation is provided by the EnSite Array™ catheter distributed by St. Jude Medical, Inc.

FIG. 10 illustrates one embodiment of an instrument assembly (708) configured to include force sensing capabilities. The instrument assembly (708), which includes a sheath catheter (602) and a guide catheter (604) co-axially positioned in the sheath catheter (602), is coupled to and maneuvered by a robotic surgical system (not shown), and configured to provide the necessary information (e.g., force feedback, etc.) to a system operator or surgeon in order to determine whether the catheter (604), and in particular the electrode (606), is in adequate contact with the interior wall surface of the heart (600). As described above, such information may be used to generate multiple three-dimensional maps or model of the surface or wall of the heart. For example, one three-dimensional map or model may show the interior wall of the heart (600) from points or locations acquired from the electrode (606) when the tip of the catheter (604) is in actual contact with the surface or wall of the heart (600) based on the force feedback information.

As is described in greater detail herein, additional three-dimensional maps or models may be produced from points or locations acquired from the electrode (606) when the catheter (604) is slightly off of the surface and not quite contacting the surface or wall of the heart (600), but in sufficiently close proximity of the wall of the heart (600). Force sensing may be used in combination with a catheter equipped with one or more electrodes to plot, mark, or trace the surface or wall of the heart, with differing levels of applied force to produce various three-dimensional maps or models of the surface of the heart. In addition, force sensing data may be used to verify or calibrate the three-dimensional maps produced by a 3-D mapping system (302). That is, force sensing may be used to determine if a 3-D map is produced from points that are on the actual surface of the heart (600) or from points that are slightly off of the surface of the heart (600) based on force feedback information.

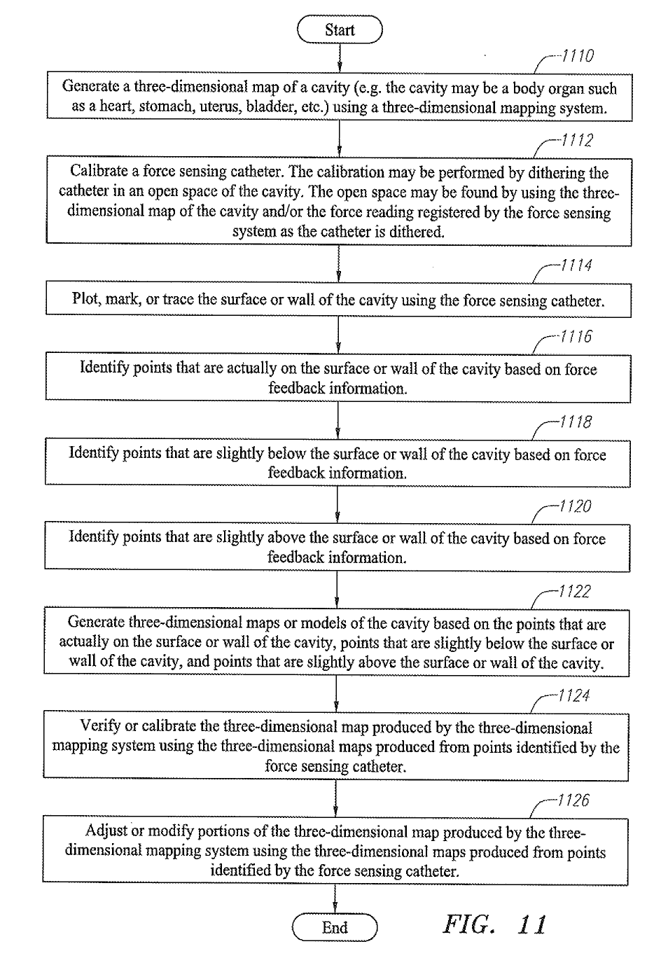

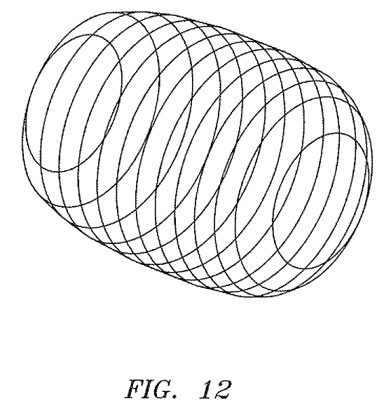

FIG. 11 is a process flowchart for verifying and/or calibrating a three-dimensional map or portions of a three-dimensional map produced by a conventional three-dimensional mapping system by employing embodiments of the disclosed inventions using force sensing. The process begins (step 1110) by using a conventional three-dimensional mapping system (e.g., the EnSite NavX™ Navigation & Visualization Technology from St. Jude Medical, Inc.) to generate a three-dimensional map of the tissue surface walls defining a cavity. The cavity may be a body organ such as a heart, stomach, uterus, bladder, prostate, etc. At step 1212, a force sensing system calibrates a force sensing catheter to establish a force baseline associated with a particular environment in a particular cavity or organ in a patient. By way of non-limiting example, the calibration may be performed by dithering the catheter in an open space of the cavity, wherein the open space is located using the three-dimensional map of the cavity and/or the force reading registered by the force sensing system as the catheter is dithered. The dithering process substantially “breaks off” or eliminates or substantially reduces any frictional adhesion of the catheter to any supporting elements or structures (e.g., a sheath catheter). This is needed because the force sensing system will, for the most part, register the force caused by the frictional or resistant force or baseline force caused by the environment in the cavity (e.g., blood or other fluids in the cavity, etc.). Such baseline forces in various environments or cavities may be factored or calibrated into the force sensing system. Once the initial calibration process is completed, the force sensing system is be capable of identifying or registering the forces asserted or exerted to or from the surface of the cavity.

At step 1114, one or more force sensing catheters plot, mark, or trace the surface or wall of the cavity (described in greater detail herein). At step 1116, the force sensing system registers the forces transmitted from the force sensing catheter as the surface or wall of the cavity is plotted, marked, or traced and points that are actually on the surface or wall of the cavity are identified based on force feedback information. For example, a force threshold may be determined to indicate when the tip of the force sensing catheter is just touching the surface of the cavity (e.g., a substantially negligible force reading). Accordingly, a force reading above the force threshold may indicate that the tip of the force sensing catheter has pushed onto the surface of the cavity (surfaces of cavities in a patient are typically comprised of compliant tissues), such that the surface of the cavity may have deflected from its normal state. Furthermore, a force reading below the force threshold may indicate that the tip of the force sensing catheter may not be touching the surface of the cavity.

At step 1118, the force sensing system registers the forces transmitted from the force sensing catheter as the surface or wall of the cavity is plotted, marked, or traced and points that are slightly below the surface or wall of the cavity are identified based on force feedback information. At step 1120, the force sensing system registers the forces transmitted from the force sensing catheter as the surface or wall of the cavity is plotted, marked, or traced and points that are slightly above the surface or wall of the cavity are identified based on force feedback information. At step 1122, the system generates a three-dimensional map or models of the tissue walls defining the cavity based on the collected position data points that are determined (based on the sensed force) to be actually on the cavity wall surface, along with points that are slightly below the surface or wall of the cavity, and points that are slightly above the surface or wall of the cavity.

Thus, in accordance with one embodiment, the process for mapping an area of internal body tissue includes: (a) maneuvering a distal end portion of an elongate instrument within an interior body region; (b) determining a position of the instrument distal end portion within the body region; (c) sensing a force applied to the instrument distal end portion at the determined position; (d) repeating acts (a) to (c) for a multiplicity of determined positions of, and sensed forces applied to, the instrument distal end portion within the interior body region; and (e) processing the respective determined positions and sensed forces for the multiplicity of determined positions to generate a geometric rendering of an internal body tissue surface located within the interior body region. In one embodiment, processing the respective determined positions and sensed forces to generate a geometric rendering of the internal body tissue surface comprises identifying a plurality of determined positions of the instrument distal end portion within the interior body region at which a substantially same amount of applied force is detected. FIG. 12 depicts a generated geometric rendering of the body cavity tissue surface constructed in accordance with this embodiment.

At step 1124, a comparison (i.e., to verify or calibrate) the three-dimensional map(s) produced by the conventional three-dimensional mapping system during operation 1110 with the three-dimensional map(s) produced from points identified or determined by the force sensing catheter, and in operation 1126, appropriate adjustments or modifications are made to the three-dimensional map produced by the conventional three-dimensional mapping system.

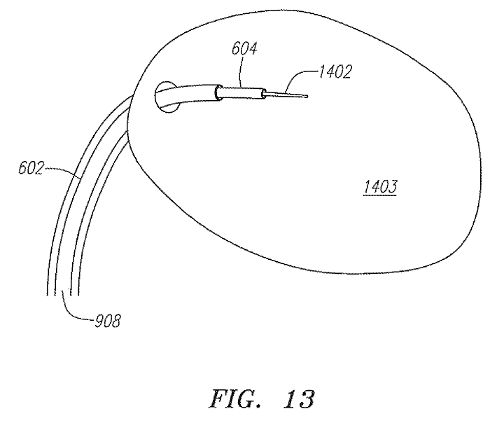

FIG. 13 illustrates the distal end portion of a medical instrument assembly (908) including a sheath catheter (602), a guide catheter (604), and a surgical instrument such as a needle or knife (1402) positioned within an interior region of a body cavity (1403). As is well-known, a fluoroscope may capture an image of the instrument assembly (908) and produce a two-dimensional image of the instrument assembly inside the cavity. In the field of electrophysiology, standard views of an anatomy are anterior posterior view, right anterior oblique view, right anterior view, posterior anterior view, left anterior view, and left anterior oblique view.

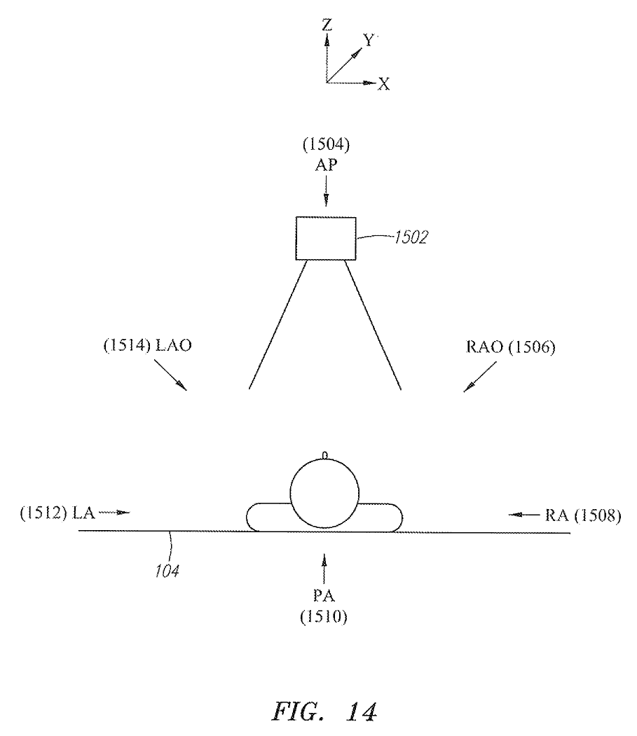

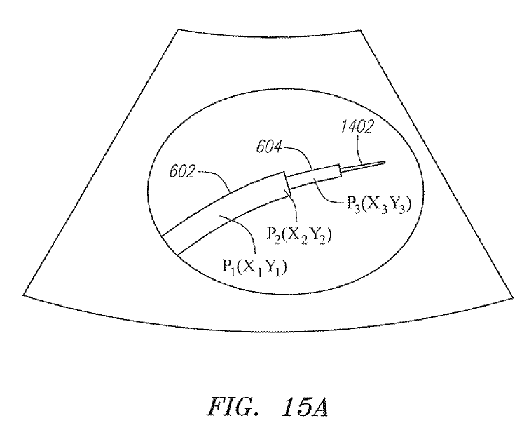

FIG. 14 illustrates the views of an anatomy as may be captured by a fluoroscope (1502). For example, a view of an anatomy or portion of an anatomy taken substantially from above a patient who is lying on an operation table (104) at about 0 degrees or about twelve O'clock position is the anterior posterior (AP) (1504) view. A projection of the AP view may be on an x-y plane. A view of an anatomy or portion of an anatomy taken from about a rightward position above a patient who is lying on an operation table (104) in the range of about 30 degrees and about 60 degrees or in the range of about one O'clock position and about two O'clock position is a right anterior oblique (RAO) (1506) view. A view of an anatomy or portion of an anatomy taken from about a right position to a patient who is lying on an operation table (104) in the range of about 60 degrees and about 90 degrees or in the range of about two O'clock position and about three O'clock position is a right anterior (RA) (1508) view. A projection of the RA view may be on a y-z plane. A view of an anatomy or portion of an anatomy taken from a lower position below a patient who is lying on an operation table (104) in the range of about 150 degrees and about 210 degrees or in the range of about five O'clock position and about seven O'clock position is the posterior anterior (PA) (1510) view. A projection of the PS view may be on an x-y plane. A view of an anatomy or portion of an anatomy taken from about the left position to a patient who is lying on an operation table (104) in the range of about 270 degrees and about 300 degrees or in the range of about nine O'clock position and about ten O'clock position is the left anterior (LA) (1512) view. A view of an anatomy or portion of an anatomy taken from about the leftward position above a patient who is lying on an operation table (104) in the range of about 300 degrees and about 330 degrees or in the range of about ten O'clock position and about eleven O'clock position is the left anterior oblique (LAO) (1514) view.

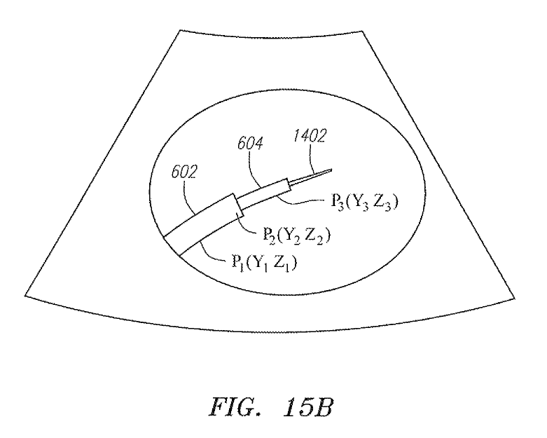

For purposes of illustration, FIGS. 15A and 15B depict some of the steps of registering an object using fluoroscopy to determine the spatial positions of the object. For example, in FIG. 15A, the instrument assembly (108) in a cavity is registered in the AP view to determine the x-y coordinates for three points on the instrument assembly (108), wherein P1 has the coordinates (x₁, y₁), P2 has the coordinates (x₂, y₂), and P3 has the coordinates (x₃, y₃). In FIG. 15B, the instrument assembly (108) in a cavity (1602) is registered in the RA view to determine the y-z coordinates for three points on the instrument assembly (108), wherein P1 has the coordinates (y₁, z₁), P2 has the coordinates (y₂,z₂), and P3 has the coordinates (y₃, z₃). The registrations of points P1, P2, and P3 in the AP and RA views may be combined to determine the x-y-z coordinates for points P1, P2, and P3. In this example, x-y-z coordinates for P1 is x₁, y₁, zᵢ, P2 is X₂, y₂, z₂, and P3 is x₃, y₃, z₃. Accordingly, the spatial positions for points P1, P2, and P3 are determined.

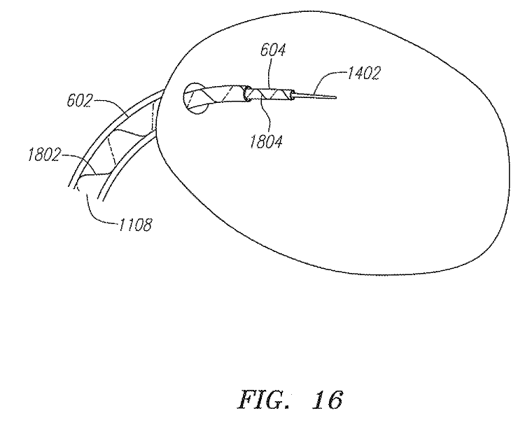

FIG. 16 illustrates an instrument assembly (1108) coupled with one or more optical fiber sensors (1802, 1804, etc.) configured with Bragg gratings. The optical sensors are configured to enable the determination of the spatial shape and position of the instrument assembly, such as the coordinate of points on the instrument assembly. A detailed discussion of spatial shape and position sensing with optical sensors is set forth in each of the above-incorporated U.S. patent application Ser. Nos. 11/690,116, 12/106,254, and 12/192,033, as well as in above-incorporated U.S. Provisional Patent Application No. 61/003,008.

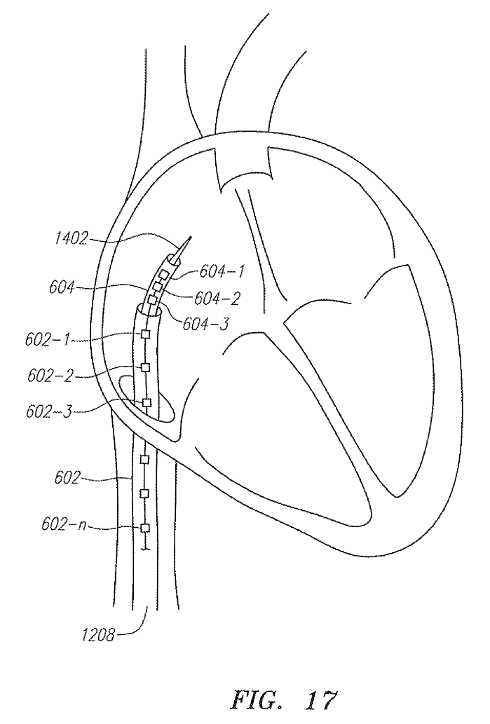

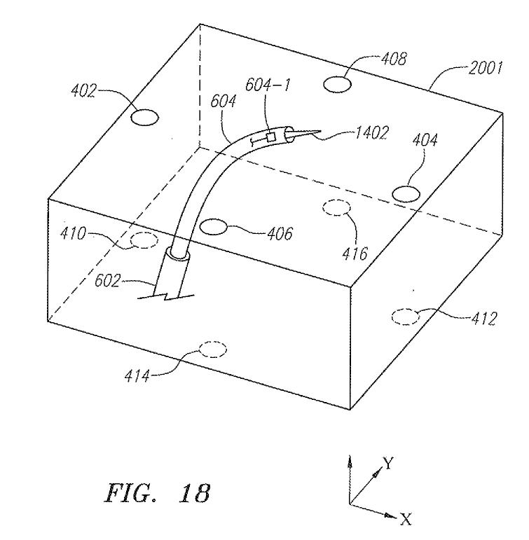

FIG. 17 illustrates a distal end of an instrument assembly (1208) coupled with electrodes (604-1, 604-2, 604-3, . . . , 604-n, and 602-1, 602-2, 602-3, . . . , 602-n) in an electrical field to determine the locations or positions of the electrodes on the instrument assembly. For example, the locations or positions of the electrodes on the instrument assembly (1208) may be determined based on the voltage potential of the electrodes in the electrical field and the voltage potential of the electrodes coupled to the instrument assembly. For example, as illustrated in FIG. 18, electrode 604-1 is located in a spatial volume or cavity (2001), which may be an organ in a body of a patient. Electrodes (402, 404, 406, 408, 410, 412, 414, 416, etc.) may be configured around or near the spatial volume or cavity (2001) and produce a modulating electrical field in the cavity (2001).

The electrical potential of the guide electrode (604-1) on the guide catheter (604) of the instrument assembly (1208) may be compared to the electrical potentials to the various pairs of electrodes (e.g., 402 and 404, 406 and 408, 402 and 410, etc.) to determine the x, y, and z positions or coordinates of the guide electrode (604-1). For example, the guide electrode (604-1) may have an electrical potential reading of 10 mV, and the electrical field electrodes (402 and 404) may have respective electrical potential readings of 5 mV and 15 mV. Accordingly, the guide electrode (604-1) may be located substantially in the middle of the field electrode (402) and field electrode (404). Alternatively, the field electrodes (402 and 404) may have respective y-coordinates of y=5 and y=15, while the guide electrode (604-1) may have a y-coordinate of y=10. Following a similar process, the spatial position or three-dimensional coordinates of the guide electrode (604-1) may be determined. Executing a similar process for all the electrodes (604-1, 604-2, 604-3, . . . , 604-n, and 602-1, 602-2, 602-3, . . . , 602-n), the spatial positions or three-dimensional coordinates for all the electrodes coupled to the instrument assembly may be determined.

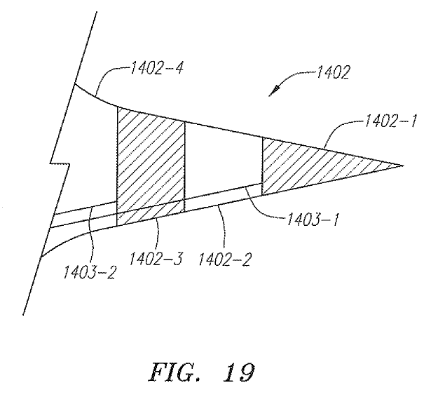

FIG. 19 illustrates a portion of the needle (1402) on instrument assembly 1208, wherein certain sections of the needle are configured to behave or function as electrodes. For example, certain sections (1402-1, 1402-3, etc.) may be fabricated from a conductive material (e.g., stainless steel, Nitinol™, etc.) and connected to circuitry by an insulated conductor (1403-1, 1403-2, etc.) function as electrodes, while other sections (1402-2, 1402-4, etc.) may be fabricated from a non-conductive material (e.g., polymeric material, urethane, poly-urethane, nylon, etc.) insulating the conductive sections. The electrodes of the needle (1402) may be used as electrodes in an electrical field as discussed above to determine the spatial position or three-dimensional coordinates of the needle (1402). Because the needle (1402) may be configured with multiple electrodes, the spatial position or three-dimensional coordinates of the sections of the needle (1402) may be determined.

The aforementioned three-dimensional maps or models as discussed in the foregoing detailed description may be combined with the spatial positions or three-dimensional coordinate information as discussed above, such that the relative position of an object in a volume or cavity may be determined. Such information as applied to minimal invasive procedures would be very useful to a surgeon who is performing such procedures on a patient. In other words, the surgeon is able to “see” where a surgical instrument is located in a patient's body, where a surgical instrument is located in an organ where the minimal invasive procedure is being performed, and where or how close the surgical instrument is located to the spot where the operation has to be executed.

In some situations, the reference frame or coordinate system of the three-dimensional model of the volume or cavity of the patient may not align with the reference frame or coordinate system (e.g., as associated with fluoroscopy, optical fiber position and shape sensing, electrodes in an electrical field, etc.) of the objects (such as an instrument assembly (108)) that are advanced or navigated into the volume or cavity of the patient. As such, in some situations it may be useful to align the reference frame or coordinate system of the three-dimensional model of the volume or cavity of the patient with the reference frame or coordinate system of the objects that are advanced or navigated into the volume or cavity of the patient. Various reference frame or coordinate system alignment, transformation, translation, rotation, etc. methodologies (e.g., Euler, etc.) may be used to align the reference frames or coordinate systems. For example, in order to align the two reference frames or coordinate systems, one or both of the coordinate systems may be translated in one or more of the axes (x-axis, y-axis, and z-axis). In addition, one or both of the coordinate systems may be rotated along one or more of the axes (x-axis, y-axis, and z-axis) to achieve alignment. Alignment or re-alignment the reference frames or coordinate systems may need to be repeated throughout the minimally invasive operation to ensure accurate spatial and positional information.

In one embodiment, a robotic surgical system may include programmable instructions to operate the instrument driver to drive, advance, steer and operate any tools or instruments coupled to the guide catheter and/or sheath catheters of the instrument assembly to be within the boundary of one of the three-dimensional maps. In another embodiment, the guide catheter and/or sheath catheter may include control wires that are coupled to circuitry (e.g., parallel circuits, etc.) that is capable of identifying when any one of the control wires is broken.

Thus, in accordance with a main embodiment of the disclosed the invention, a robotic instrument system includes a controller, an instrument driver in communication with the controller, and an elongate instrument coupled to the instrument driver, the instrument driver configured to manipulate a distal end portion of the guide instrument in response to control signals generated by the controller. A force sensing system may be coupled directly to the instrument (e.g., embedded in a wall portion at or near the instrument distal end), and the robotically driven instrument may itself be a working instrument, e.g., having mapping and/or ablation electrodes or other operative elements on its distal end. In other embodiments, the robotically controlled instrument is a guide instrument having a working lumen in which a separate working instrument, such as a mapping and/or ablation catheter, is coaxially disposed, in which case the force sensing system is preferably coupled directly to the working instrument. The force sensing system may comprises one or more sensors (e.g., strain gauges) embedded in or otherwise coupled to the distal end portion of the respective instrument, or at some other location. In one embodiment, the force sensing system is the above described “dithering” system that is coupled to a proximal portion of a working instrument that extends proximally from the robotic guide instrument.

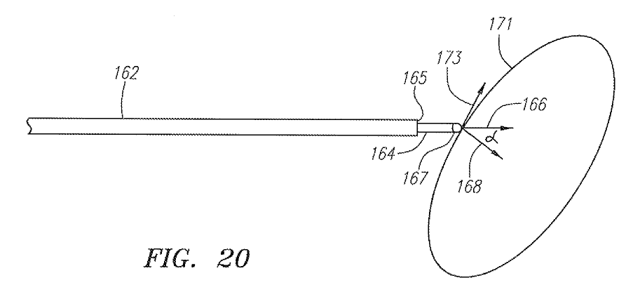

The force sensing system may be a unidirectional, for example, only sensing forces applied substantially normal to a longitudinal axis of the instrument; or it may be a multi-directional force sensing system. In the case of unidirectional force sensing along the axis of the respective elongate instrument, the system processor may still calculate an estimated applied force normal to a tissue surface even where the respective force sensing instrument is pressed against the tissue surface at a non-orthogonal angle, as long as the relative positions of the instrument and tissue surface are known. By way of illustration, FIG. 20 depicts a robotic guide instrument 162 approaching a tissue surface 171 at a non-orthogonal trajectory, with a distal end portion 166 of a working instrument extending out of a distal opening 165 of the guide instrument 162 and contacting the tissue surface 171 at a non-orthogonal angle. A unidirectional force sensing system associated with the working instrument 164 senses an applied force from the tissue surface 171 on the working instrument distal end 167 along a longitudinal axis of the working instrument distal end portion. Because the position of the guide instrument 162 and/or working instrument 164 relative to the tissue surface 171 is known/previously registered by the system, the system processor can readily determine the angle of incidence a between the longitudinal axis of the working instrument 164 and a plane normal to the tissue surface 171 at the contact location, e.g., by fitting one of a set of triangles to the image data. From that information, a component 168 of the axial force 166 in a direction normal to the tissue surface at the contact location may be calculated, as well as a component 173 of the axial force 166 in a direction tangential to the tissue surface at the contact location.

Thus, the force sensing system is configured to generate force signals responsive to a force applied by a tissue surface to a distal end portion of the respective guide or working instrument normal to the tissue surface. Similarly, a position determining system is part of or otherwise integrated with the robotic instrument system, the positioning system including one or more position sensors that generate position data indicative of a position of the distal end portion of the respective guide or working instrument (hereinafter, “sensing instrument”) where applicable). A processor, which may incorporated as part of the system controller, or a separate processor in communication with the controller, is operatively coupled to the respective force sensing and position sensing systems. The processor generates and causes to be displayed on a display associated with the system a geometric rendering (or “map”) of an internal body tissue surface based at least in part upon sensed forces applied to the distal end of the sensing instrument as it is maneuvered within an interior region of a body containing the body surface.

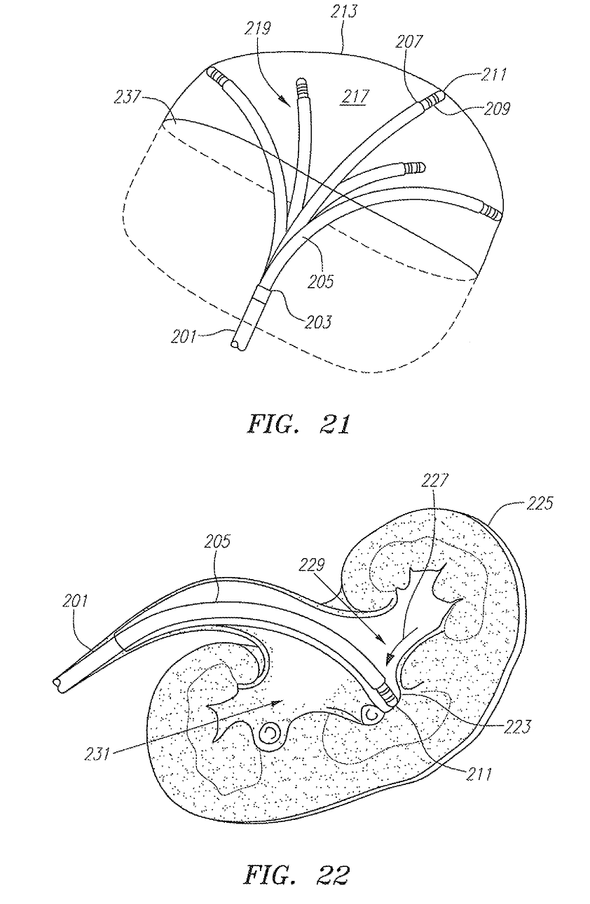

By way of illustration, FIG. 21 depicts a distal end portion of a robotic guide instrument 205 extending from a distal opening 203 of a more proximally positioned (and relatively stationary) sheath instrument 201 into an open interior body region, or anatomical workspace 217 (e.g., a heart chamber) to be mapped. A distal tip portion of a working instrument 209, e.g., a mapping and/or ablation catheter, is carried (and thus maneuvered) by the guide instrument 205, and is shown extending out of a distal opening 207 of the guide instrument 205, i.e., by retraction of the guide instrument 205 relative to the working instrument 209, extension of the working instrument 209 relative to the guide instrument 205, or some of each. The guide instrument 205 is initially positioned under operator control to a location approximately centered within the anatomical workspace 217. Thereafter, as indicated by the illustrated distributed positions of the guide instrument 205 within the interior body region 217 (indicated by arrow 219), the guide instrument 205 is maneuvered about the interior region to collect respective force sensing and position data relating to the distal tip 211 of the working instrument 209.

Maneuvering of the guide instrument 205 within the anatomical workspace 217 may be accomplished under operator control, or automated under system control, or some of each. By way of non-limiting example, the system may be configured to maneuver the guide instrument 205 along a predetermined set of trajectories that explore the workspace 217 based on its approximate dimensions obtained, also by way of non-limiting example, from an imaging system, such as fluoroscopic image data. The trajectories are preferably calculated by the system in order to obtain an adequate amount of force and position data to construct a reliable structural map of the tissue surface boundary 213 (or a portion thereof) of the workspace 217. Such trajectories may be configured to obtain a three-dimensional grid of points (“tapper-mapper”) or to stay along fixed radial lines (e.g., line 237 in FIG. 21) to form a radial grid (e.g., as seen in FIG. 12), or may be some combination of radial and grid of points, e.g., such as a planar grid (radial slices).

Even if an automated process is employed to obtain the necessary force/position mapping data, the system preferably displays a representation of the anatomical workspace 217 to the operator so that the operator may maneuver the guide instrument 205 to portions of the anatomy to be mapped that are not captured by the automated mapping process. It will be appreciated that, as the working instrument tip 211 is moved about the workspace 217, it will periodically come into contact with the tissue surface 213, whether by purposeful or random trajectory of the guide instrument 205, allowing for accumulation of respective force and position data adequate to generate a reliable structural map of the tissue surface 213. It will also be appreciated that when obtaining the force and associated position data for a heart chamber of a beating heart, well-known filtering or heart rhythm gating techniques must be employed. If contact with the tissue surface is not sensed at the limit of the guide workspace (i.e., the limit to which the guide instrument 205 can be safely extended along a particular trajectory), the system preferably records that no contact was detected. When all of the trajectories have been traversed by the system, a three-dimensional map of the explored workspace is displayed to the operator, preferably also showing any areas where the guide instrument 205 reached its own workspace limit with no resistance, so that the operator may take those locations into account for further planning purposes.

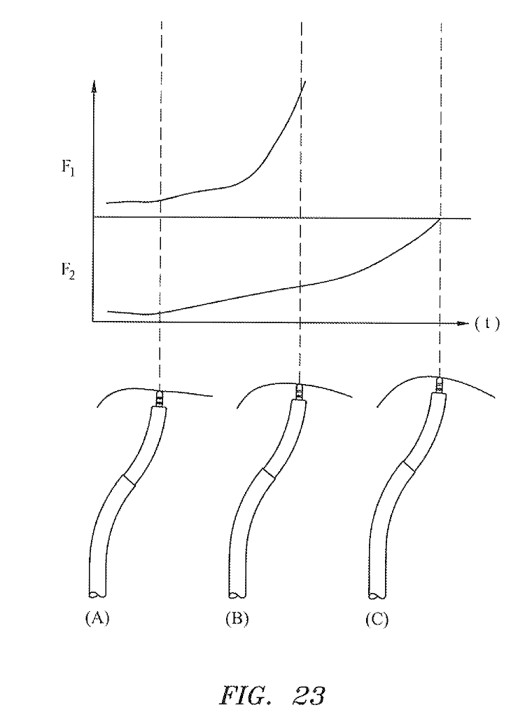

By way of further example, FIG. 22 depicts the distal end portion of the guide instrument 205 positioned within the interior region of a kidney 225 during a “mapping” procedure. In particular, the distal tip 211 of the working instrument 209 is shown extending from the guide instrument 205 and in contact with a tissue surface 223 of the interior region of the kidney 225. As indicated by arrow 227, the guide instrument 205 is moving along a lateral trajectory, approximately normal to the tissue surface 223, from an “already mapped” area 219 to an “unmapped” area 231, collecting the necessary force and position data along the way. In this embodiment, rather than “swirling” the tip 211 about the region, or otherwise employing a predetermined set of sensing instrument trajectories as described above, until sufficient surface contact points are identified, a substantially constant “contact force” is maintained against the tissue surface 223 by the working instrument tip 211 as the guide instrument 205 moves the tip 211 along the tissue surface 223 for some number of approximately parallel trajectories, until sufficient position data is obtained to determine the desired structural map. It will be appreciated that other surface contact verification sensors (e.g., capacitive, optical, impedance, local tissue activity detection) may be employed in addition to force sensing techniques.

In one variation available for certain regions and anatomical workspaces in the body, especially where the fluid in the organ chamber to be mapped is relatively clear, such as in the kidney shown FIG. 22, the attending physician or other operator (collectively “operator”) may first identify a region of abnormality (e.g., a growth, protrusion, calcified tissue, etc.) on a surface (either internal or external) of an organ inside the patient's body, for example, by way of direct visualization through the optical system (endoscope built into the catheter, a semiconductor based digital imaging unit built-into the distal portion of the robotic catheter, etc.) integrated on the robotic guide instrument, or other imaging modality (X-Ray, CT Scan, MRI). The operator then drives the distal tip of the robotic instrument towards the abnormality with the assistance of the direct visual guidance. Through the controller, the physician can manually drive the distal instrument tip to palpate the region of the abnormality using a working instrument carried by or built into the robotic instrument, visually inspecting the abnormality and at the same time mapping out the plasticity (e.g., hardness of the abnormal tissue in relation to the adjacent tissue) of the region of abnormality. In an alternative embodiment, the imaging unit may be carried on a separate robotic arm from the force sensing robotic arm, as the above mapping procedure is being conducted by the arm carrying the force sensor.

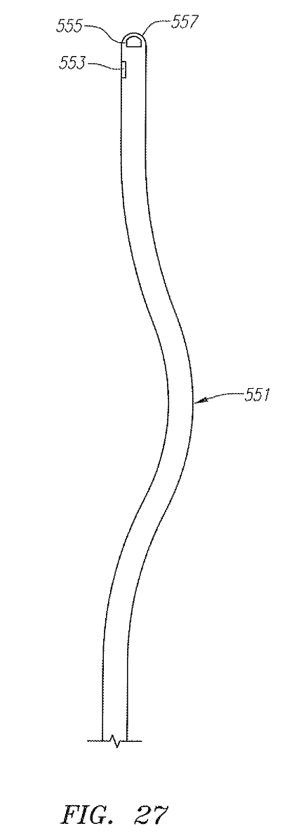

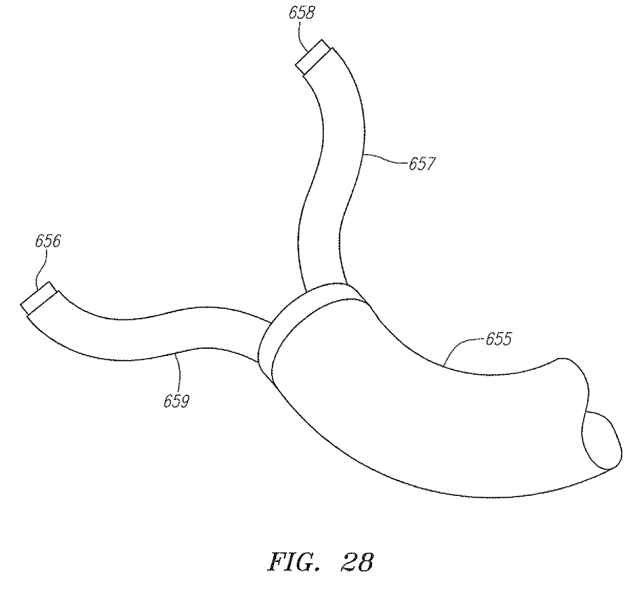

In another variation, the mapping process is conducted automatically. The physician first defined a region of abnormality to be mapped on an image model created by the computer in the control counsel. A commend is then given to the computer in the control counsel to direct the distal tip of the robotic catheter to scan/touch/palpate the surface of the defined region to be mapped. In one example, the computer controls a catheter carrying the force sensor to dither over the surface of the defined region. In another example, the computer controls the catheter to glide the force sensor on the catheter over the defined region. Once the command to automatically map the plasticity/tissue-characteristic of the define region is given, the physician may monitor the mapping process through one or more of the following mechanisms or a combination thereof: (1) direct visualization through the imaging modality integrated within the robotic catheter; (2) the force sensor output being display on monitor at the control counsel; (3) real time imaging (e.g., fluoroscope, MRI, ultrasound, etc.) of the organ; (4) a computer model (e.g., computer generated cartoon) of the organ's internal space along with a moving catheter model indicating the position of the catheter within the organ's internal space. Further embodiments of a robotic instrument system that can be used for such alternate embodiments are shown in FIGS. 27-28. FIG. 27 illustrates another embodiment of a distal end portion of a robotically controlled catheter instrument 551, including both a force sensor 553 and an image capture device (e.g., camera) 555 in its distal tip. Alternatively, a direct visualization endoscopic lens may be provided instead of the image capture device. FIG. 28 illustrates still another embodiment of a robotic instrument system, including a pair of robotic guide instruments 657, 659 extending from an access sheath 655, one of the robotic guide instruments 657 having a force sensor 658 in its distal tip, and the other robotic arm 659 having an image capture device in its distal tip. Alternatively, a direct visualization endoscopic lens may be provided instead of the image capture device.

It will be appreciated that the boundaries of a generated and displayed geometric map necessarily depends on the threshold level of force applied to the distal end of the sensing instrument at which the system (whether based on operator input or otherwise automated) determines the instrument tip is contacting the tissue surface. In one embodiment, an amount of force indicative of tissue surface contact may be derived by obtaining sample data by maneuvering the sensing instrument directly into one or more locations on the tissue surface to be mapped up to a maximum threshold force level. While the sensing instrument tip remains in an open space and not contacting any tissue structure within the workspace, a relatively low level of force is applied to the instrument tip, e.g., just that exerted by the patient's blood pressure along with inherent system friction when navigating in the blood vessel system. However, as the instrument approaches and contacts a tissue surface, the applied force will ramp up, with a steepness of the ramp (or curve) being a function of the tissue surface stiffness or compliance. Thus, by viewing and/or analyzing one or more sample force curves, the operator (if done manually) and/or the system processor can identify a point along the curve in which it is readily apparent that tissue surface contact has been made.

For purposes of illustration, FIG. 23 depicts simplified unidirectional force curves F₁ and F₂ obtained as the sensing instrument distal end approaches, contacts, and continues to push against respective tissue surface locations that differ in their stiffness. In particular, force curves F₁ and F₂ both indicate that surface contact is made at approximately time “A” in the respective working instrument trajectory towards and against the respective surface locations. Both force curves thereafter continue in the same shape/progression, indicating similar compliance of the outermost tissue at the respective surface locations. However, prior to time “B” in the respective trajectories, force curve F₁ shows a marked increase in slope compared to force curve F₂, indicating that the tissue at the surface location corresponding to force curve F₁ is less compliant than the tissue at the surface location corresponding to force curve F₂, which does not show any marked change in slope until approaching time “C” of its trajectory. Thus, while the very initial contact force may be similar for most all tissue surface locations, depending on the compliance of a particular area (e.g., scar tissue versus healthy muscle or even fatty tissue), it can greatly vary just under the immediate surface.

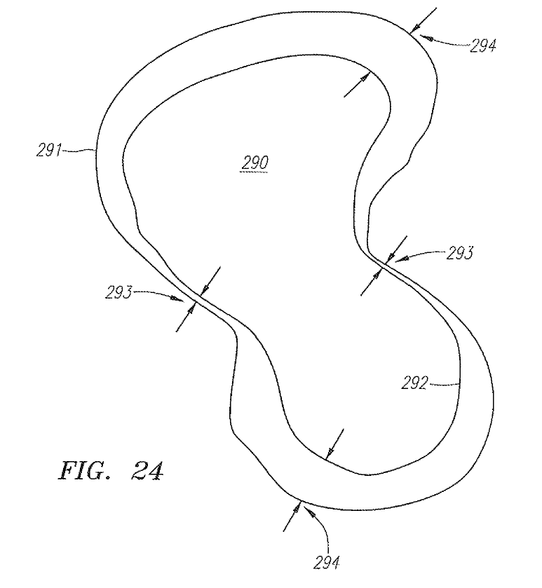

As previously stated, the boundaries of a geometric tissue surface map generated in accordance with the force sensing techniques described herein will necessarily vary depending on the selected threshold level of applied force chosen as representative of “tissue contact”. Thus, it may be beneficial to chose a force level that is clearly over (or at least slightly over) the actual level indicating initial surface contact in order to achieve consistent results from map to map. Another approach, shown in FIG. 24, is to generate a multi-force level map, akin to a topology map, wherein at least two different boundaries are identified and depicted based on a corresponding at least two different force levels. As with a “single” (uniform) force level map, the data for a multi-force map can be collected in multiple ways, including by allowing the sensing instrument tip to move freely about the interior region of the anatomical workspace and into the tissue surface (to differing depths) to be mapped, until enough position data is collected for each of the at least two different force levels. In variations of this approach, the sensing instrument trajectories can be implemented to maintain a force level above, below, or in between operator or system selected minimum and/or maximum threshold force levels for purposes of maximizing quality position data collection for surface mapping purposes. In another embodiment, a substantially uniform first force level is maintained while collecting position data for a first surface map, and then the process is repeating at a higher (or lower) substantially uniform second force level. Obviously, a same approach for the collection of correlated position and force data can be used to construct a single surface map, or it may be desired in some instances to generate more than two force level maps for a more fine-tuned understanding of the tissue compliance as a function of depth, and the disclosed inventions are not so limited to mapping just one or two force levels.

Regardless of the data collection technique that is employed, the processor can produce a graphic rendering of respective “tissue surfaces” generated based on differing force levels, similar to a topography map. By way of illustration, FIG. 24 depicts a multi-force level map of a tissue surface circumscribing a body cavity region 290, including a first (inner) tissue surface boundary 292 determined based upon a first plurality of identified positions within the interior body region at which a first, substantially same amount of force was detected on the distal end portion of the sensing instrument, and a second tissue surface boundary 291 determined based upon a second plurality of identified positions within the interior body region at which a second, substantially same amount of force was detected on the distal end portion of the sensing instrument, the second force level being great than the first force level, such that the second boundary 291 circumscribes a larger area (volume if three-dimensional) than does the first boundary 293.

One aspect of this embodiment is that areas in the tissue surface having a markedly different compliance are readily detected based on the relative spacing between the first and second surface boundaries, 293 and 291. In particular, at locations 294, the spacing between the two force level boundaries is relatively wide, indicating a corresponding relatively high tissue compliance; whereas the spacing between the two force level boundaries is very slight at locations 293, indicating a corresponding low compliance (i.e., high stiffness) of the tissue at those locations. This difference in tissue compliance (stiffness) can be indicative of different types of tissue at the respective locations, e.g., scar tissue versus healthy muscle or even fatty tissue. In one embodiment, the system processor is configured to automatically determine a characteristic of an area of the surface tissue based on the relative spacing between respective force-level surface boundaries, and in particular a characteristic based on tissue compliance.

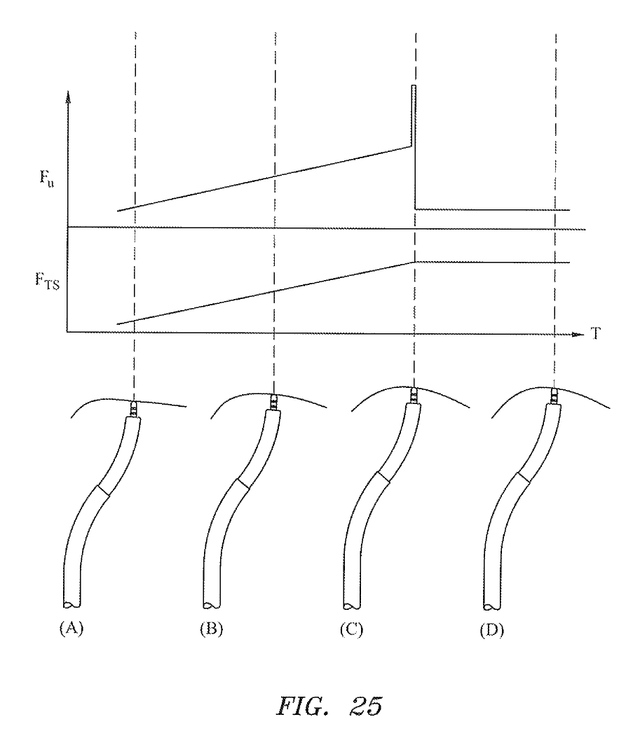

It may be desirable to provide force-feedback (e.g., through a haptic operator input interface), along with the automatic limiting and stabilization of an applied force exerted on the tissue surface during a mapping procedure. Operation of one embodiment having these features is shown in FIG. 25, which depicts a first force curve, F_{TS}, representing an actual applied to a tissue surface as the sensing instrument is pressed against the tissue surface over a period of time (from “A” to “D”). A second force curve is also depicted, Fu, which represents a relative “force” applied to the operator by a haptic user interface by the system controller, i.e., a resistance imposed by the interface to further movement of the instrument tip into the tissue surface. At time “A”, contact with the tissue surface has just occurred (or is about to), and relatively little force is applied on the tissue surface and user interface. The particular tissue surface compliance is depicted as increasing linearly between time “A” and time “B”, also reflecting a corresponding linear increase in the amount of force on the tissue surface, by the advancing instrument tip, and to the user interface, by the system controller. This linear increase continue until time “C”, at which a maximum threshold force level has been reached by the instrument, and the system automatically prevents any additional movement of the instrument into the tissue surface that may increase the applied force (reflected by the frozen horizontal force line F_{TS} starting at time “C”). In order to alert the system operator that the maximum force level has been reached, the controller causes the user interface to impart a “detent” or other sensation to the operator, reflected in the force felt by the operator, F_{U}, at time “C”), after which the user interface is disabled by the controller to prevent the operator from causing any further forward motion of the instrument into the tissue surface, as reflected in the minimal horizontal force line, F_{U}, following time “C”).

It should be appreciated that the force applied by the instrument in the forgoing example illustrated in FIG. 25 may be operator selected or automatically selected by the system. In both the former and later case, the operator may increase or decrease the applied force, as desired, during a mapping and/or tissue compliance diagnostic procedure. Either way, if the system applies the “detent” (or other haptic signal) to the user interface device to signal that the maximum allowable force was reached, the user interface is preferably disabled and the system takes over servoing the instrument at the safe instrument force level until the operator reengages the input device, rejecting such disturbances as respiration, etc. Optionally, if the system senses that the instrument distal end has moved beyond a certain limit in an effort to reach and/or maintain a selected threshold force on the target tissue surface, the system may stop servoing to the force and instead servo to position only. It should further be appreciated that other types of operator feedback may also be contemplated, such as audio and/or visual signals (or alarms), as well as written messages to the operator or color coding used in the system display, e.g., if the force exceeds a desired amount or is too low. Furthermore, visual, audio, tactile and/or motion feedback may be utilized in place of or in combination with the “detent” sensation (or other haptic signal) to provide user with feedback regarding the amount force been applied on the surface of the tissue or indicate a pre-defined threshold of force has been achieved. For example, instead of the “detent” sensation being applied, as shown in FIG. 25, the system may provide a short burse of vibration in place of the “detent” sensation. Audio (e.g., a ring tone or buzzer etc.) or visual cue (e.g., flashing cartoon of the catheter in the displayed computer model) may also be provided at the same time to alert the user that the maximum threshold force has been reach.

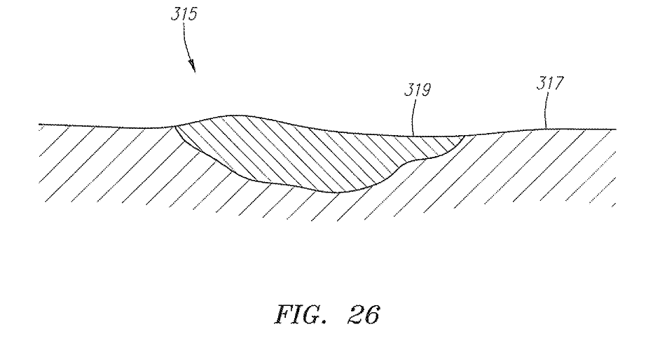

The processor is preferably configured to determine a characteristic of tissue (e.g., tissue stiffness or compliance) at a location on the tissue surface based on a sensed force applied to the distal end portion of the instrument, as the instrument is maneuvered against the tissue surface at the respective location(s). As previously described with respect to the various embodiments disclosed herein, a display may be coupled to the system processor for displaying geometric renderings of respective internal body tissue surfaces and passageways, for example, wherein regions of the body tissue area in the map having differences in tissue compliance are visually highlighted. By way of example, FIG. 26 is a side view of a generated tissue surface map 315, in which an area of tissue 319 having been identified as having relatively higher or lower compliance/stiffness than the surround tissue surface 317 is visually highlighted. In this manner, the system can assist the operator with identifying tissue anomalies in the tissue surface based on a sensed force or forces applied to the instrument distal end portion as it is maneuvered against the tissue surface in embodiments of the disclosed inventions, including determining and displaying approximate boundaries of the tissue anomaly on the tissue surface. Once identified, embodiments of the system will provide for treatment during the same session, including using a treatment mechanism (e.g., an ablation electrode) carried on the same sensing instrument that is used to perform the mapping and anomaly detection process.

In one such embodiment, treating the tissue anomaly includes one or more of determining approximate boundaries of the tissue anomaly on the tissue surface; displaying approximate boundaries of the tissue anomaly on a graphic rendering of the tissue surface; delivering treatment energy to the anomaly; delivering a treatment substance to the anomaly; and displaying an area of the anomaly that has been treated on a graphic rendering of the tissue surface. Again, one or both of delivering treatment energy to the anomaly and delivering a treatment substance to the anomaly are performed using the instrument.

While multiple embodiments and variations of the many aspects of the disclosed invention have been described herein, such description is provided for purposes of illustration only. Many combinations and permutations of the disclosed embodiments are useful in minimally invasive medical diagnosis and intervention, and the disclosed inventions are configured to be flexible and adaptable. In particular, the foregoing illustrated and described embodiments of the disclosed inventions are suitable for various modifications and alternative forms, and it should be understood that the disclosed inventions generally, as well as the specific embodiments described herein, are not limited to the particular forms or methods disclosed, but also cover all modifications, alternatives, and equivalents as defined by the scope of the appended claims. Further, the various features and aspects of the illustrated embodiments may be incorporated into other embodiments, even if not so described herein, as will be apparent to those skilled in the art. In addition, although the description describes data being mapped to a three dimensional model, data may be mapped to any mapping or coordinate system, including two dimensional, static or dynamic time-varying (temporal) map, coordinate system, model, image, etc. All directional references (e.g., upper, lower, upward, downward, left, right, leftward, rightward, top, bottom, above, below, vertical, horizontal, clockwise, counterclockwise, etc.) are only used for identification purposes to aid the reader's understanding of the disclosed inventions without introducing limitations as to the position, orientation, or applications of the invention. Joining references (e.g., attached, coupled, connected, and the like) are to be construed broadly and may include intermediate members between a connection of elements (e.g., physically, electrically, optically as by an optically fiber, and/or wirelessly connected) and relative physical movements, electrical signals, optical signals, and/or wireless signals transmitted between elements. Accordingly, joining references do not necessarily infer that two elements are directly connected in fixed relation to each other.

1. A medical instrument system, comprising:
a controller;
an instrument driver in communication with the controller;
an elongate instrument coupled to the instrument driver, the instrument driver configured to manipulate a distal end portion of the instrument in response to control signals generated by the controller;
a force sensor associated with the instrument, wherein the force sensor generates force signals responsive to a force applied to the distal end portion of the instrument;
a position determining system which generates position data indicative of a position of the distal end portion of the instrument;
a processor operatively coupled to the force sensor and position determining system, and configured to process the respective force signals and position data to generate a geometric rendering of an internal body tissue surface based at least in part upon sensed forces applied to the distal end of the instrument as it is maneuvered within an interior region of a body containing the body surface; and
a display coupled to the processor for displaying the geometric rendering of an internal body tissue surface.

2. The system of claim 1, wherein the processor is configured to determine a characteristic of tissue at a location on the tissue surface based on a sensed force applied to the distal end portion of the instrument as the instrument is maneuvered against the tissue surface at the respective location.

3. The system of claim 2, wherein the determined tissue characteristic is a measure of tissue stiffness or compliance.

4. The system of claim 3, wherein the processor causes regions of the body tissue area in the map having differences in tissue compliance to be visually highlighted on the display.

5. The system of claim 1, wherein the force sensor is coupled to the distal end portion of the instrument.

6. They system of claim 5, wherein the force sensor comprises a unidirectional force sensor that senses force applied substantially normal to a longitudinal axis of the instrument.

7. (canceled)

8. The system of claim 1, wherein the processor is configured to generate the graphic rendering of the tissue surface by identifying a plurality of determined positions of the instrument distal end portion within the interior body region at which a substantially same amount of applied force is detected.

9. The system of claim 1, wherein the graphic rendering of the tissue surface includes a first tissue surface boundary determined based upon a first plurality of locations within the interior body region at which a first substantially same amount of force is detected on the distal end portion of the instrument, and a second tissue surface boundary determined based upon a second plurality of locations within the interior body region at which a second substantially same amount of force greater than the first amount is detected on the distal end portion of the instrument.

10. (canceled)

11. A medical instrument system, comprising:
a controller;
an instrument driver in communication with the controller;
a guide instrument coupled to the instrument driver, the instrument driver configured to manipulate a distal end portion of the guide instrument in response to control signals generated by the controller;
a working instrument carried by the guide instrument and having a distal tip portion;
a force sensor associated with the working instrument, wherein the force sensor generates force signals responsive to a force applied to a distal end portion of the working instrument;
a position determining system which generates position data indicative of a position of the distal end portion of the working instrument;
a processor operatively coupled to the force sensor and position determining system, and configured to process the respective force signals and position data to generate a geometric rendering of an internal body tissue surface based at least in part upon sensed forces applied to the distal end of the working instrument while it is extended out of a distal opening of the guide instrument and maneuvered by the guide instrument within an interior region of a body containing the body surface; and
a display coupled to the processor for displaying the geometric rendering of an internal body tissue surface.

12. (canceled)

13. The system of claim 11, wherein the working instrument comprises an ablation or mapping catheter.

14-24. (canceled)

25. A method of mapping an area of body tissue, comprising:
(a) maneuvering a distal end portion of an elongate instrument within an interior body region;
(b) determining a position of the instrument distal end portion within the body region;
(c) sensing a force applied to the instrument distal end portion at the determined position;
(d) repeating acts (a) to (c) for a multiplicity of determined positions of, and sensed forces applied to, the instrument distal end portion within the interior body region;
(e) processing the respective determined positions and sensed forces for the multiplicity of determined positions to generate a geometric rendering of an internal body tissue surface located within the interior body region; and
(f) displaying the generated geometric rendering of the body tissue surface.

26. The method of claim 25, wherein processing the respective determined positions and sensed forces to generate a geometric rendering of the internal body tissue surface comprises identifying a plurality of determined positions of the instrument distal end portion within the interior body region at which a substantially same amount of applied force is detected.

27. The method of claim 25, wherein the geometric rendering of the internal body tissue surface includes a first tissue surface boundary determined based upon a first plurality of locations within the interior body region at which a first substantially same amount of force is detected on the instrument distal end portion, and a second tissue surface boundary generated based upon a second plurality of locations within the interior body region at which a second substantially same amount of force greater than the first amount is detected on the instrument distal end portion.

28. The method of claim 27, further comprising determining a characteristic of an area of tissue in the tissue surface based on a relative spacing between the first and second surface boundaries.

29. A method of mapping an area of body tissue, comprising:
(a) maneuvering a distal end portion of an elongate instrument within an interior body region;
(b) sensing forces applied to a distal end portion of the instrument;
(c) determining a position of the instrument distal end portion when a sensed force applied thereto is indicative of tissue surface contact; and
(d) repeating acts (a) to (c) for a multiplicity of determined tissue surface contact positions within the interior body region;
(e) processing the multiplicity of determined positions to generate a geometric rendering of an internal body tissue surface located within the interior body region; and
(f) displaying the generated geometric rendering of the body tissue surface.

30. The method of claim 29, further comprising displaying a representation of the interior body region to an operator to facilitate initial positioning of the instrument distal end within the region under the control of the operator prior to obtaining of the multiplicity of determined positions.

31. The method of claim 29, wherein the instrument is coupled to an instrument driver configured to manipulate the instrument distal end portion in response to control signals generated by a controller.

32. The method of claim 31, wherein the controller causes the instrument distal end to be maneuvered along a determined set of trajectories based on the interior body region.

33. The method of claim 32, wherein the interior body region is selected from a group comprising
a heart chamber,
an anatomical workspace at least partially surrounding an organ exterior surface,
an interior of an organ,
an interior of the gastro-intestinal tract.

34-36. (canceled)

37. The method of claim 29, further comprising identifying a tissue anomaly in the tissue surface based on a sensed force or forces applied to the instrument distal end portion as it is maneuvered against the tissue surface.

38. The method of claim 37, further comprising determining and displaying approximate boundaries of the tissue anomaly on the tissue surface.

39. The method claim 37, further comprising treating the tissue anomaly.

40. The method of claim 39, wherein treating the tissue anomaly comprises one or more of
determining approximate boundaries of the tissue anomaly on the tissue surface;
displaying approximate boundaries of the tissue anomaly on a graphic rendering of the tissue surface;
delivering treatment energy to the anomaly;
delivering a treatment substance to the anomaly; and
displaying an area of the anomaly that has been treated on a graphic rendering of the tissue surface.

41. The method of claim 40, wherein one or both of delivering treatment energy to the anomaly and delivering a treatment substance to the anomaly are performed using the instrument.

42. A medical instrument system, comprising:
a controller having a user interface for receiving operator input commands;
an instrument driver in communication with the controller;
an elongate instrument coupled to the instrument driver, the instrument driver configured to manipulate a distal end portion of the instrument in response to control signals generated by the controller at least partially in response to received operator commands;
a force sensor associated with the instrument, wherein the force sensor generates force signals responsive to a force applied to the distal end portion of the instrument;
a processor operatively coupled to the force sensor and controller, and configured to process the respective force signals to generate applied force data based at least in part upon sensed forces applied to the distal end of the instrument; and
a display coupled to the processor for displaying the applied force data.

43. The system of claim 42, wherein the operator commands include an applied force limit.

44. A method of diagnosing and/or treating internal body tissue, comprising maneuvering a distal end portion of an elongate instrument against an internal body tissue surface until either (i) sensing that a threshold level of force is being applied by the instrument distal end against the tissue surface, or (ii) the instrument distal end is extended to or beyond a determined movement limitation.

45-50. (canceled)

51. A medical instrument system, comprising:
an elongate instrument;
a controller configured to selectively actuate one or more motors operatively coupled to the instrument to thereby selectively move the instrument;
a force sensor associated with the instrument, wherein the force sensor generates force signals responsive to a force applied to the distal end portion of the instrument;
a processor operatively coupled to the force sensor and controller, and configured to process the respective force signals to generate applied force data based at least in part upon sensed forces applied to the distal end of the instrument; and
a haptic input device in communication with the controller and configured for generating instrument motion commands in response to a directional movement of the input device,
wherein the controller transmits signals to the input device to cause the input device to impart a detectable resistance to movement of the input device corresponding to an actual amount of force being applied against the instrument distal end portion.

52. A method of diagnosing and/or treating internal body tissue, comprising:
maneuvering a distal end portion of an elongate instrument against an internal body tissue surface;
sensing an axial force vector applied by the body surface to the instrument distal end portion;
determining an angle of incidence at which the instrument distal end portion is contacting the body surface; and
projecting, based on the sensed axial force vector and determined contact angle of incidence, a component of the axial force in a direction normal to the tissue surface at the contact location.

53. The method of claim 52, further comprising projecting a component of the axial force vector in a direction tangential to the tissue surface at the contact location.
